(19) United States
(12) Patent Application Publication
(10) Pub. No.: US 2009/0281115 A1
(43) Pub. Date: Nov. 12, 2009

(54) INHIBITORS OF C-KIT AND USES THEREOF

(75) Inventors: William Bornmann, Missouri City, TX (US); David Maxwell, Pearland, TX (US); Ashutosh Pal, Houston, TX (US); Zhenghong Peng, Missouri City, TX (US); Zeev Estrov, Houston, TX (US)

Correspondence Address: BAKER BOTTS, LLP, 910 LOUISIANA, HOUSTON, TX, 77002-4995, US

(73) Assignee: Board of Regents, The University of Texas System, a Texas University, Austin, TX (US)

(21) Appl. No.: 12/307,001

(22) PCT Filed: Jun. 29, 2007
(86) PCT No.: PCT/US07/72555

§ 371 Date: Dec. 30, 2008

Related U.S. Application Data
(60) Provisional application No. 60/806,385, filed on Jun. 30, 2006.
(60) Provisional application No. 60/807,381, filed on Jul. 14, 2006.

(51) Int. Cl. A61K 31/496; C07D 471/04; A61K 31/437; A61P 35/00; C12N 9/99

(52) U.S. Cl. 514/253.04; 546/119; 514/303; 544/362; 435/184

(57) ABSTRACT

Compounds and methods useful as inhibitors of c-Kit are presented. Pharmaceutical compositions containing these compounds, methods of using these compounds as inhibitors of c-Kit and processes for synthesizing these compounds are also described herein.

### CROSS-REFERENCE TO RELATED APPLICATIONS

This application claims priority to U.S. Pat. App. Ser. No. 60/806,385 filed Jun. 30, 2006 and to U.S. Pat. App. Ser. No. 60/807,381 filed Jul. 14, 2006. Applications are incorporated by reference herein in their entirety.

### FIELD OF THE INVENTION

The present invention is directed to compounds that inhibit c-Kit, their design, their synthesis, and their application as a pharmaceutical for the treatment of disease.

### STATEMENT REGARDING FEDERALLY SPONSORED RESEARCH OR DEVELOPMENT

None.

### THE NAMES OF THE PARTIES TO A JOINT RESEARCH AGREEMENT

None.

### REFERENCE TO SEQUENCE LISTING

None.

### BACKGROUND OF THE INVENTION

A tyrosine kinase is an enzyme that transfers a phosphate group from ATP to a tyrosine residue in a protein. Tyrosine kinases are a subgroup of the larger class of protein kinases. Fundamentally, a protein kinase is an enzyme that modifies a protein by chemically adding phosphate groups via phosphorylation. Such modification often results in a functional change to the target protein or substrate by changing the enzyme activity, cellular location or association with other proteins. Chemically, the kinase removes a phosphate group from ATP and covalently attaches it to one of three amino acids (serine, threonine or tyrosine) that have a free hydroxyl group. Most kinases act on both serine and threonine, and certain others, tyrosine. There are also a number of kinases that act on all three of these amino acids.

Tyrosine kinases are divided into two groups: cytoplasmic proteins and transmembrane receptor kinases. In humans, there are 32 cytoplasmic protein tyrosine kinases and 48 receptor-linked protein-tyrosine kinases.

c-Kit (CD117) is a protein-tyrosine kinase and the transmembrane receptor for stem cell factor (SCF). SCF, also known as “steel factor” and “c-kit ligand,” is a polypeptide that activates bone marrow precursors of a number of blood cells. However, SCF's receptor (c-Kit) is also present on other cells. Furthermore, c-Kit is a CD molecule (cluster of differentiation molecule). CD molecules are a defined set of cell surface molecules which serve as markers on the cell surface and are recognized by specific sets of antibodies. These antibodies are used to identify the cell type, stage of differentiation and activity of a cell.

Generally, kinases are enzymes known to regulate the majority of cellular pathways, especially pathways involved in signal transduction or the transmission of signals within a cell. Because protein kinases have profound effect on a cell, kinase activity is highly regulated. Kinases can be turned on or off by phosphorylation (sometimes by the kinase itself -cis- phosphorylation/autophosphorylation) and by binding to activator proteins, inhibitor proteins or small molecules.

Deregulated kinase activity is a frequent cause of disease, particularly cancer where kinases regulate many aspect that control cell growth, movement and death. For example, neoplastic transformation in which multiple genetic defects such as translocation, mutations within oncogenes and the like, have been implicated in the development of leukemia. Many of these genetic defects have been identified as key components of signaling pathways responsible for proliferation and differentiation.

For example, mutations of the activation loop of c-Kit are associated with certain human neoplasms including systemic mast cell disorders, seminoma, acute myelogenous leukemia (AML), gastrointestinal stromal tumors (GISTs) and hypopigmentary disorders. AML is the most common form of leukemia and the most common cause of leukemia death. Activating mutations of receptor tyrosine kinases are associated with distinct genetic subtypes in AML.

Systemic mastocytosis, for example, has been found to be associated with activating mutations of the c-Kit gene corresponding to amino acid Asp-816 (D816), which mutation has been used as a tracking marker to elucidate the clonal nature of mastocytosis. Mast cells derive from a hematopoietic lineage that is dependent on c-Kit signaling for growth, differentiation, and survival. Mast cells are found in excessive numbers in tissues in a heterogeneous group of disorders collectively known as mastocytosis. KIT-D816 mutations are associated with impaired event-free and overall survival.

Recently, the small-molecule tyrosine kinase inhibitor imatinib mesylate (STI571, GLEEVEC™) has been identified as potent inhibitor of wild-type (WT) c-Kit and certain mutant c-Kit isoforms. For metastatic GIST, imatinib has become current the standard of care for treating patients. c-Kit mutations in the interstitial cells of Cajal in the disgestive tract reportedly explain the efficacy of imatinib in the management of the gastrointestinal stromal tumors (GISTs) malignancies. Specifically, c-Kit activity is believed to provide growth and survival signals to GIST.

Notwithstanding, the activation loop mutations of c-Kit involving the codon for D816 that are typically found in AML, systemic mastocytosis, and seminoma are insensitive to imatinib mesylate (IC50>5-10 micromol/L). In addition, acquired c-Kit activation loop mutations can be associated with imatinib mesylate resistance in GIST. Indeed, imatinib mesylate binding and c-Kit inhibition has been shown to be abrogated by the c-Kit domain I missense mutation Val654Ala.

Furthermore, distinct forms of tyrosine kinase domain (TKD), juxtamembrane domain, exon 8, and internal tandem duplication (ITD) mutations of c-Kit are present in about 46% of core binding factor leukemia (CBFL) patients.

The current lack of diagnostic assays and markers predictive of sensitivity to c-Kit inhibitors has slowed the assessment of drugs targeting this kinase. Therefore, a need exists for compounds useful in treating disease associated with deregulation of the c-Kit kinase and methodology for designing such additional compounds associated with c-kit kinase and other tyrosine protein kinase related disorders.

### BRIEF SUMMARY OF THE INVENTION

Novel compounds and pharmaceutical compositions that inhibit c-Kit have been found together with methods of structurally designing such compounds, methods of synthesizing and methods of using the compounds including methods for of inhibiting c-Kit disorders in a patient by administering the compounds.

The present invention discloses a class of compounds useful in treating c-Kit-mediated disorders and conditions, defined by the structural Formula I:

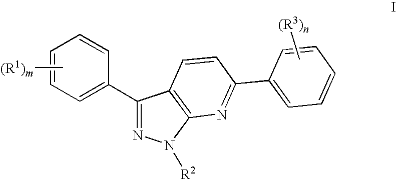

wherein m and n is an integer from 1 to 5;
R¹ is independently halogen, alkyl, alkoxycarbonyl, alkoxy, hydrogen, or cyano, any of which may be optionally substituted;
R² is selected from the group consisting of hydrogen, acyl, alkenyl, alkoxyalkyl, alkoxycarbonyl, alkoxycarbonylalkyl, alkyl, alkylaminocarbonyl, alkylcarbonylalkyl, alkylthioalkyl, alkylsulfinylalkyl, alkynyl, aminoalkyl, aminocarbonylalkyl, aryl, arylsulfonyl, aralkyl, carboxyalkyl, cycloalkyl, haloalkyl, heteroaryl, heteroaralkyl, or hydroxyalkyl, any of which may be optionally substituted; and
R³ is independently hydrogen, acetamido, acyl, alkenyl, alkoxyalkyl, alkoxycarbonyl, alkoxycarbonylalkyl, alkyl, alkylaminocarbonyl, alkylcarbonylalkyl, alkylthioalkyl, alkylsulfinylalkyl, alkynyl, aminoalkyl, aminocarbonyl, aminocarbonylalkyl, aryl, arylsulfonyl, arylcarbonyl, fused pyrrole, aralkyl, carboxyalkyl, cycloalkyl, haloalkyl, heteroaryl, heteroaralkyl, hydroxyalkyl, or phenol any of which may be optionally substituted;
wherein when n=2, R³ is not CONHEt and Me; and
when n=3, R³ is not F, Me, and CONHEt;
F, Me and

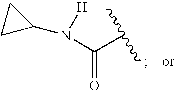

F, Me and

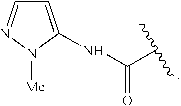

Compounds according to the present invention possess useful c-Kit inhibiting or modulating activity and may be used in the treatment or prophylaxis of a disease or condition in which c-Kit plays an active role. Thus, in the broad aspect, the present invention provides for pharmaceutical compositions comprising one or more compounds of the present invention together with a pharmaceutically acceptable carrier as well as methods of making and using the compounds and compositions. The present invention provides methods for treating a c-Kit-mediated disorder in a patient in need of such treatment comprising administering to said patient a therapeutically effective amount of a compound or composition according to the subject invention. The present invention also contemplates the use of compounds disclosed herein for use in the manufacture of a medicament for the treatment of a diseases or condition ameliorated by the inhibition of c-Kit.

### BRIEF DESCRIPTION OF THE SEVERAL VIEWS OF THE DRAWINGS

FIG. 1. Schematic showing signal transduction by stem cell factor (SCF) acting on c-Kit with subsequent intracellular signaling cascades.

FIG. 2. Schematic showing crystal structure of c-Kit bound to GLEEVEC® (STI-571, IMATINIB®) in the c-Kit ATP binding pocket.

FIG. 3. Flowchart showing in silico strategy for selection of candidate c-Kit inhibitors.

FIG. 4. Compound 3-{(4-Methyloxyphenyl)-1H-pyrazolo[4,3-b]pyridin-6-yl}acetanilide as designed and synthesized herein shown docked in silico in the ATP binding pocket of c-Kit.

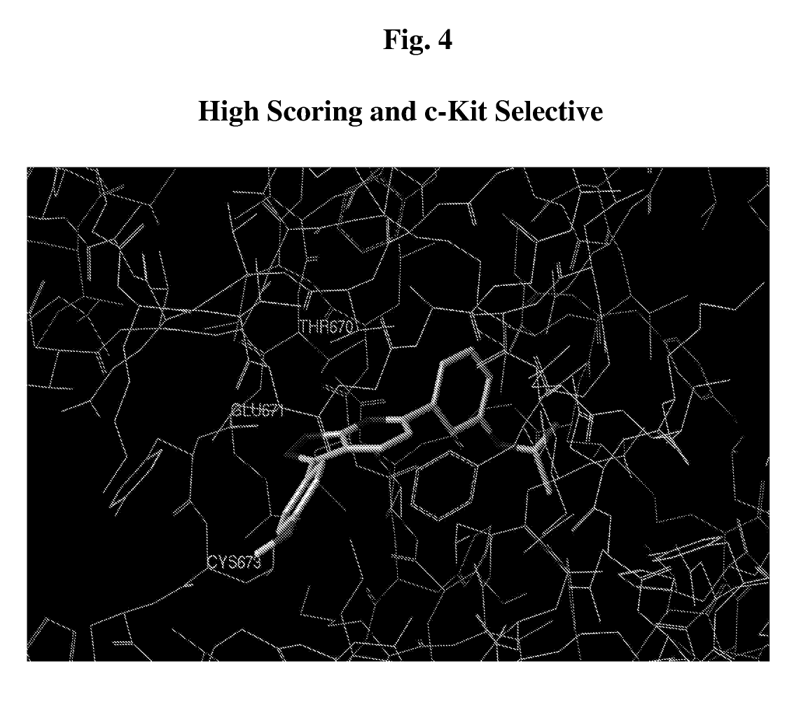

FIG. 5. Concentration-response plot for several c-Kit inhibitors as set forth herein.

FIG. 6. Dose-dependent results of cell viability studies using an MTT-based assay of an AML cell line, OCL-AML3, expressing c-Kit in the presence of various concentrations of antagonists as provided herein. This cell line is resistant to IMATINIB and plateaued with Dasatinib.

FIG. 7. Dose-dependent results of cell viability using an MTT-based assay of an AML cell line, OCIM2, which, when uninduced by SCF, expresses c-Kit at a much lower level compared to the OCI/AML3 cell line. The reduced effect on the OCIM2 cell line as compared to the data of FIG. 6 indicates that the antagonist has specificity towards c-Kit.

FIG. 8. Dose-dependent results of stem cell factor induction of c-Kit in the OCIM2 cell line. The OCI-AML3 line constitutively expresses c-Kit and is essentially unresponsive to induction.

FIG. 9. Dose-dependent inhibition of c-Kit kinase by antagonist 1-[3-(4-fluoro)-1H-pyrazolo[3,4-b]pyridin-6-yl]-3,5-dimethoxy]benzene in the OCIM2 cell line with and without stem cell factor induction (50 ng/mL SCF).

FIG. 10. Concentration response plot for GLEEVEC® in the AML cell lines OCIM2 (uninduced) and OCI-AML3 (expressing c-Kit). These cell lines are resistant to prior art compound IMATINIB (GLEEVEC®) at concentrations at which antagonists as set forth herein possess inhibitory activity.

FIG. 11. Concentration response plot of inhibitory activity of 1-[3-(4-fluoro)-1H-pyrazolo[3,4-b]pyridin-6-yl]-3,5-dimethoxy]benzene as set forth herein as compared to prior art compounds Dasatinib (BMS) and GLEEVEC® (IMATINIB) in the OCI-AML3 line expressing c-Kit.

FIG. 12. The effect of compound 1-[3-(4-fluoro)-1H-pyrazolo[3,4-b]pyridin-6-yl]-3,5-dimethoxy]benzene on two systemic mastocytosis cell lines; cell line HCM1.1 has wild-type amino acid Asp at position 816 of c-Kit while cell line HCM1.2 has a mutation at amino acid position 816. Assay data confirming this compound inhibitory activity and selectivity for the cell line containing mutated c-Kit.

FIG. 13. The effect of compound 1-[3-(4-fluoro)-1H-pyrazolo[3,4-b]pyridin-6-yl]-3,5-dimethoxy]benzene on HMC1.2 cells. The cells were incubated with compound 1-[3-(4-fluoro)-1H-pyrazolo[3,4-b]pyridin-6-yl]-3,5-dimethoxy]benzene for thirty (30) minutes.

FIG. 14. The effect of compound 1-[3-(4-fluoro)-1H-pyrazolo[3,4-b]pyridin-6-yl]-3,5-dimethoxy]benzene on OCI/AML3 cells.

FIG. 15. 1-[3-(4-fluoro)-1H-pyrazolo[3,4-b]pyridin-6-yl]-3,5-dimethoxy]benzene induces the accumulation of OCI/AML3 in Sub-Go phase of the cell cycle.

FIG. 16. The effect of compound 1-[3-(4-fluoro)-1H-pyrazolo[3,4-b]pyridin-6-yl]-3,5-dimethoxy]benzene on proliferation in OCIm2 and OCI/AML3.

FIG. 17. OCIm2 and OCI/AML3 AML cells produce SCF expressed on their surface SCF receptor (c-Kit; CD117).

FIG. 18. 1-[3-(4-fluoro)-1H-pyrazolo[3,4-b]pyridin-6-yl]-3,5-dimethoxy]benzene inhibits OCI/AML3 (but not OCIM2) colony-forming cell proliferation

FIG. 19. SCF neutralizing antibodies (P=0.001) inhibit OCI/AML3 colony proliferation and their inhibitory effect is completely reversed by exogenous SCF.

FIG. 20. SCF and SCF neutralizing antibodies do not affect OCI/M2 colony proliferation.

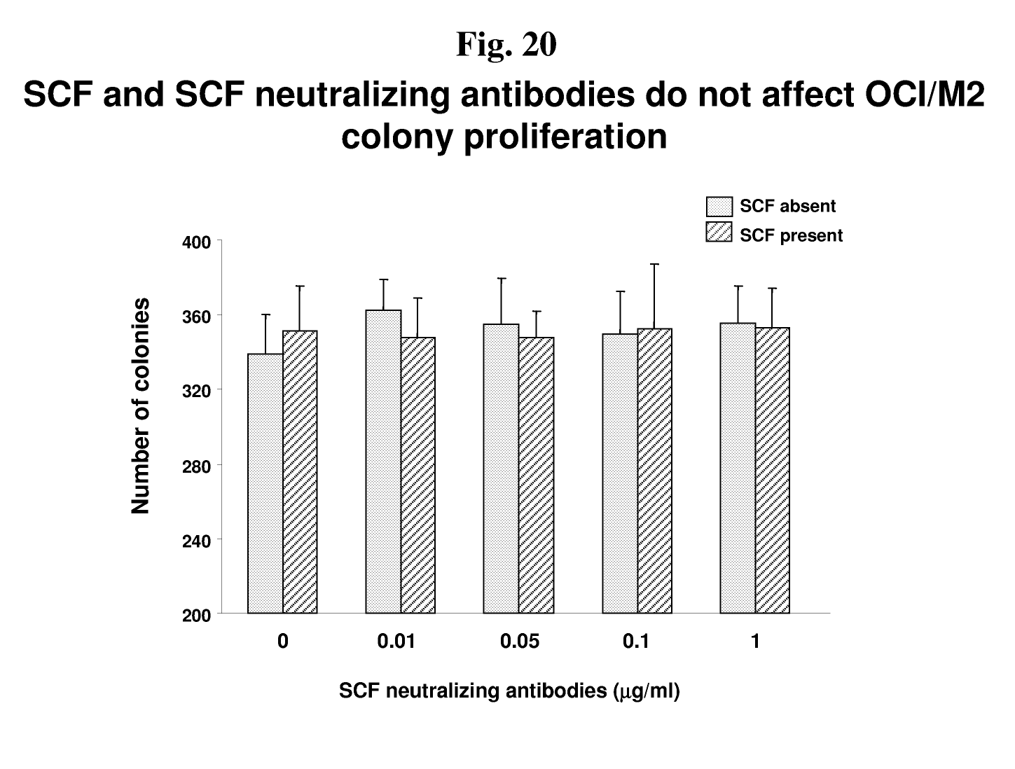

FIG. 21. Inhibition of cell proliferation by 1-[3-(4-fluoro)-1H-pyrazolo[3,4-b]pyridin-6-yl]-3,5-dimethoxy]benzene in OCIM2, OCI/AML3 and HMC1.1.

FIG. 22. Inhibition of cell proliferation by 1-[3-(4-fluoro)-1H-pyrazolo[3,4-b]pyridin-6-yl]-3,5-dimethoxy]benzene in the absence and presence of SCF.

FIG. 23. Inhibition of cell proliferation of AML1, AML2, AML3, AML4, AML5 and AML6 by 1-[3-(4-fluoro)-1H-pyrazolo[3,4-b]pyridin-6-yl]-3,5-dimethoxy]benzene in the presence of SCF.

FIG. 24. Inhibitory concentration of OCIM-3 as percent of control of BMS-354825, Gleevec, 1-[3-(4-fluoro)-1H-pyrazolo[3,4-b]pyridin-6-yl]-3,5-dimethoxy]benzene and SCF

FIG. 25. Dose response in OCI/AML3 cell line.

### DETAILED DESCRIPTION OF THE INVENTION

In certain embodiments, the compounds of the present invention have structural Formula II:

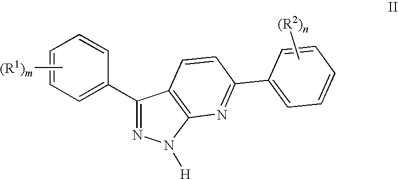

wherein m and n is an integer from 1 to 5;
R¹ is independently hydrogen, halogen, alkyl, ester, alkoxy, hydrogen, or cyano, any of which may be optionally substituted; and
R² is independently hydrogen, acetamido, acyl, alkenyl, alkoxyalkyl, alkoxycarbonyl, alkoxycarbonylalkyl, alkyl, alkylaminocarbonyl, alkylcarbonylalkyl, alkylthioalkyl, alkylsulfinylalkyl, alkynyl, aminoalkyl, aminocarbonyl, aminocarbonylalkyl, aryl, arylsulfonyl, arylcarbonyl, fused pyrrole, aralkyl, carboxyalkyl, cycloalkyl, haloalkyl, heteroaryl, heteroaralkyl, hydroxyalkyl, or phenol any of which may be optionally substituted;
wherein when n=2, R² is not CONHEt and Me; and
when n=3, R² is not F, Me, and CONHEt;
F, Me and

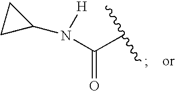

F, Me and

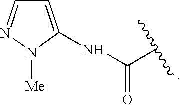

Specific compounds of particular interest consist of compounds and pharmaceutically-acceptable salts, esters and prodrugs thereof as follows:
3-{(4-Methyloxyphenyl)-1H-pyrazolo[4,3-b]pyridin-6-yl}acetanilide;
3-[3-(4-Fluoro-3-methylphenyl)-1H-pyrazolo[3,4-b]pyridin-6-yl]acetanilide;
3-[3-(4-Fluorophenyl)-1H-pyrazolo[3,4-b]pyridin-6-yl]acetanilide;
3-[3-(4-Chloro-3-fluorophenyl)-1H-pyrazolo[3,4-b]pyridin-6-yl]acetanilide;
3-[3-(4-Ethoxycarbonyl)-1H-pyrazolo[3,4-b]pyridin-6-yl]acetanilide;
4-[3-(4-Ethoxycarbonyl)-1H-pyrazolo[3,4-b]pyridin-6-yl]-2,6-dimethyl]phenol;
[4-{3-(4-Chloro-3-fluorophenyl)-1H-pyrazolo[3,4-b]pyridin-6-yl}-2,6-dimethyl]phenol;
1-[3-(4-Ethoxycarbonyl)-1H-pyrazolo[3,4-b]pyridin-6-yl]-3,5-dimethoxy]benzene;
1-[3-(4-fluoro-3-methyl)-1H-pyrazolo[3,4-b]pyridin-6-yl]-3,5-dimethoxy]benzene;
1-[3-(4-fluoro)-1H-pyrazolo[3,4-b]pyridin-6-yl]-3,5-dimethoxy]benzene;
1-[3-(4-chloro-3-fluoro)-1H-pyrazolo[3,4-b]pyridin-6-yl]-3,5-dimethoxy]benzene;
1-[3-(4-methoxy)-1H-pyrazolo[3,4-b]pyridin-6-yl]-3,5-dimethoxy]benzene;
4-[3-(4-ethoxycarbonyl)-1H-pyrazolo[3,4-b]pyridin-6-yl]-N,N-dimethyethyldiaminocarbonyl]benzene;
3-[3-(4-ethoxycarbonyl)-1H-pyrazolo[3,4-b]pyridin-6-yl]-]benzamide;
3-[3-(4-Fluorophenyl)-1H-pyrazolo[3,4-b]pyridin-6-yl]benzamide;
2-[3-(4-Chloro-3-fluorophenyl)-1H-pyrazolo[3,4-b]pyridin-6-yl]phenol;
Ethyl-4-[3-(4-methyloxyphenyl)-1H-pyrazolo[3,4-b]pyridin-6-yl]benzoate;
4-{3-(4-Methyloxyphenyl)-1H-pyrazolo[3,4-b]pyridin-6-yl}acetanilide;
4-[3-(4-Ethyloxycarbonylphenyl)-1H-pyrazolo[3,4-b]pyridin-6-yl]acetanilide;
4-[3-(4-Bromo-3-fluorophenyl)-1H-pyrazolo[3,4-b]pyridin-6-yl]acetanilide;
4-[3-(4-Bromo-2-fluorophenyl)-1H-pyrazolo[3,4-b]pyridin-6-yl]acetanilide;
4-[3-(4-Fluorophenyl)-1H-pyrazolo[3,4-b]pyridin-6-yl]acetanilide;
4-[3-(4-Chloro-3-fluorophenyl)-1H-pyrazolo[3,4-b]pyridin-6-yl]acetanilide;
4-[3-(4-Cyanophenyl)-1H-pyrazolo[3,4-b]pyridin-6-yl]acetanilide;
4-[3-(3,4-Dichlorophenyl)-1H-pyrazolo[3,4-b]pyridin-6-yl]acetanilide;
4-[3-(2,4-Dichlorophenyl)-1H-pyrazolo[3,4-b]pyridin-6-yl]acetanilide;
4-[3-(4-Bromophenyl)-1H-pyrazolo[3,4-b]pyridin-6-yl]acetanilide;
4-[3-(3-Fluoro-4-methylphenyl)-1H-pyrazolo[3,4-b]pyridin-6-yl]acetanilide;
4-[3-(4-Methylphenyl)-1H-pyrazolo[3,4-b]pyridin-6-yl]acetanilide;
4-[3-(2,4-Dimethylphenyl)-1H-pyrazolo[3,4-b]pyridin-6-yl]acetanilide;
4-[{3-(3-Methyloxyphenyl)-1H-pyrazolo[3,4-b]pyridin-6-yl}-3-methyloxy]phenol;
Ethyl-4-[3-(4-methoxycarbonyl)-1H-pyrazolo[3,4-b]pyridin-6-yl]-]benzoate;
Diphenyl-4-{3-(4-ethoxycarbonylphenyl)-1H-pyrazolo[3,4-b]pyridin-6-yl}ketone;
Diphenyl-4-{3-(4-methyloxyphenyl)-1H-pyrazolo[3,4-b]pyridin-6-yl}ketone;
Diphenyl-4-{3-(4-chloro-3-fluorophenyl)-1H-pyrazolo[3,4-b]pyridin-6-yl}ketone;
Diphenyl-4-{3-(4-fluorophenyl)-1H-pyrazolo[3,4-b]pyridin-6-yl}ketone;
Diphenyl-4-{3-(3-fluoro-4-methylphenyl)-1H-pyrazolo[3,4-b]pyridin-6-yl}ketone;
Diphenyl-4-{3-(4-bromo-3-fluorophenyl)-1H-pyrazolo[3,4-b]pyridin-6-yl}ketone;
Diphenyl-4-{3-(4-bromo-2-fluorophenyl)-1H-pyrazolo[3,4-b]pyridin-6-yl}ketone;
3-[3-(2,4-Dichlorophenyl)-1H-pyrazolo[3,4-b]pyridin-6-yl]acetanilide;
3-[3-(4-Bromo-2-fluorophenyl)-1H-pyrazolo[3,4-b]pyridin-6-yl]acetanilide;
2-[3-(3,4-Dichlorophenyl)-1H-pyrazolo[3,4-b]pyridin-6-yl]acetanilide;
3-[3-(4-Bromo-3-fluorophenyl)-1H-pyrazolo[3,4-b]pyridin-6-yl]acetanilide;
3-[3-(4-Cyanophenyl)-1H-pyrazolo[3,4-b]pyridin-6-yl]acetanilide;
3-[3-(4-Bromophenyl)-1H-pyrazolo[3,4-b]pyridin-6-yl]acetanilide;
3-[3-(4-Ethylphenyl)-1H-pyrazolo[3,4-b]pyridin-6-yl]acetanilide;
3-[3-(2,4-Dimethylphenyl)-1H-pyrazolo[3,4-b]pyridin-6-yl]acetanilide;
Ethyl-4-[3-(4-ethyloxycarbonylphenyl)-1H-pyrazolo[3,4-b]pyridin-6-yl]benzoate;
Ethyl-4-[3-(4-chloro-3-fluorophenyl)-1H-pyrazolo[3,4-b]pyridin-6-yl]benzoate;
Ethyl-4-[3-(4-bromo-3-fluorophenyl)-1H-pyrazolo[3,4-b]pyridin-6-yl]benzoate;
Ethyl-4-[3-(3-fluoro-4-methylphenyl)-1H-pyrazolo[3,4-b]pyridin-6-yl]benzoate;
Ethyl-4-[3-(4-fluorophenyl)-1H-pyrazolo[3,4-b]pyridin-6-yl]benzoate;
Ethyl-4-[3-(3,4-dichlorophenyl)-1H-pyrazolo[3,4-b]pyridin-6-yl]benzoate;
Ethyl-4-[3-(4-cyanophenyl)-1H-pyrazolo[3,4-b]pyridin-6-yl]benzoate;
Ethyl-4-[3-(4-bromophenyl)-1H-pyrazolo[3,4-b]pyridin-6-yl]benzoate;
6-[3-(4-Ethyloxycarbonylphenyl)-1H-pyrazolo[3,4-b]pyridin-6-yl]-1H-indole;
Ethyl-4-[3-(4-bromo-3-fluoro)-1H-pyrazolo[3,4-b]pyridin-6-yl]benzoate;
Ethyl-4-[3-(2,4-dichloro)-1H-pyrazolo[3,4-b]pyridin-6-yl]benzoate;
6-[3-(4-Methoxyphenyl)-1H-pyrazolo[3,4-b]pyridin-6-yl]-1H-indole;
6-[3-(4-chloro-3-fluoro)-1H-pyrazolo[3,4-b]pyridin-6-yl]-1H-indole;
6-[3-(3-fluoro-4-methyl)-1H-pyrazolo[3,4-b]pyridin-6-yl]-1H-indole;
6-[3-(4-bromo-3-fluoro)-1H-pyrazolo[3,4-b]pyridin-6-yl]-1H-indole; and
6-[3-(4-Bromo-2-fluoro)-1H-pyrazolo[3,4-b]pyridin-6-yl]-1H-indole.

As used herein, the terms below have the meanings indicated.

The term “acyl,” as used herein, alone or in combination, refers to a carbonyl attached to an alkenyl, alkyl, aryl, cycloalkyl, heteroaryl, heterocycle, or any other moiety were the atom attached to the carbonyl is carbon. An “acetyl” group refers to a —C(O)CH₃ group. Examples of acyl groups include formyl, alkanoyl and aroyl radicals.

The term “acylamino” embraces an amino radical substituted with an acyl group. An example of an “acylamino” radical is acetylamino (CH₃C(O)NH—).

The term “alkenyl,” as used herein, alone or in combination, refers to a straight-chain or branched-chain hydrocarbon radical having one or more double bonds and containing from 2 to 20, preferably 2 to 6, carbon atoms. Alkenylene refers to a carbon-carbon double bond system attached at two or more positions such as ethenylene [(—CH═CH—), (—C::C—)]. Examples of suitable alkenyl radicals include ethenyl, propenyl, 2-methylpropenyl, 1,4-butadienyl and the like.

The term “alkoxy,” as used herein, alone or in combination, refers to an alkyl ether radical, wherein the term alkyl is as defined below. Examples of suitable alkyl ether radicals include methoxy, ethoxy, n-propoxy, isopropoxy, n-butoxy, iso-butoxy, sec-butoxy, tert-butoxy, and the like.

The term “alkoxyalkoxy,” as used herein, alone or in combination, refers to one or more alkoxy groups attached to the parent molecular moiety through another alkoxy group. Examples include ethoxyethoxy, methoxypropoxyethoxy, ethoxypentoxyethoxyethoxy and the like.

The term “alkoxyalkyl,” as used herein, alone or in combination, refers to an alkoxy group attached to the parent molecular moiety through an alkyl group. The term “alkoxyalkyl” also embraces alkoxyalkyl groups having one or more alkoxy groups attached to the alkyl group, that is, to form monoalkoxyalkyl and dialkoxyalkyl groups.

The term “alkoxycarbonyl,” as used herein, alone or in combination, refers to an alkoxy group attached to the parent molecular moiety through a carbonyl group. Examples of such “alkoxycarbonyl” groups include methoxycarbonyl, ethoxycarbonyl, propoxycarbonyl, butoxycarbonyl and hexyloxycarbonyl.

The term “alkoxycarbonylalkyl” embraces radicals having “alkoxycarbonyl”, as defined above substituted to an alkyl radical. In certain embodiments, alkoxycarbonylalkyl radicals are “lower alkoxycarbonylalkyl” having lower alkoxycarbonyl radicals as defined above attached to one to six carbon atoms. Examples of such lower alkoxycarbonylalkyl radicals include methoxycarbonylmethyl.

The term “alkyl,” as used herein, alone or in combination, refers to a straight-chain or branched-chain alkyl radical containing from 1 to and including 20, preferably 1 to 10, and more preferably 1 to 6, carbon atoms. Alkyl groups may be optionally substituted as defined herein. Examples of alkyl radicals include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—).

The term “alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-diethylamino and the like.

The term “alkylaminocarbonyl” as used herein, alone or in combination, refers to an alkylamino group attached to the parent molecular moiety through a carbonyl group. Examples of such radicals include N-methylaminocarbonyl and N,N-dimethylcarbonyl.

The term “alkylcarbonyl” and “alkanoyl,” as used herein, alone or in combination, refers to an alkyl group attached to the parent molecular moiety through a carbonyl group. Examples of such groups include methylcarbonyl and ethylcarbonyl.

The term “alkylidene,” as used herein, alone or in combination, refers to an alkenyl group in which one carbon atom of the carbon-carbon double bond belongs to the moiety to which the alkenyl group is attached.

The term “alkylsulfinyl,” as used herein, alone or in combination, refers to an alkyl group attached to the parent molecular moiety through a sulfinyl group. Examples of alkylsulfinyl groups include methylsulfinyl, ethylsulfinyl, butylsulfinyl and hexylsulfinyl.

The term “alkylsulfonyl,” as used herein, alone or in combination, refers to an alkyl group attached to the parent molecular moiety through a sulfonyl group. Examples of alkylsulfinyl groups include methanesulfonyl, ethanesulfonyl, tert-butanesulfonyl, and the like.

The term “alkylthio,” as used herein, alone or in combination, refers to an alkyl thioether (R—S—) radical wherein the term alkyl is as defined above. Examples of suitable alkyl thioether radicals include methylthio, ethylthio, n-propylthio, isopropylthio, n-butylthio, iso-butylthio, sec-butylthio, tert-butylthio, ethoxyethylthio, methoxypropoxyethylthio, ethoxypentoxyethoxyethylthio and the like.

The term “alkylthioalkyl” embraces alkylthio radicals attached to an alkyl radical. Alkylthioalkyl radicals include “lower alkylthioalkyl” radicals having alkyl radicals of one to six carbon atoms and an alkylthio radical as described above. Examples of such radicals include methylthiomethyl.

The term “alkynyl,” as used herein, alone or in combination, refers to a straight-chain or branched chain hydrocarbon radical having one or more triple bonds and containing from 2 to 20, preferably from 2 to 6, more preferably from 2 to 4, carbon atoms.

“Alkynylene” refers to a carbon-carbon triple bond attached at two positions such as ethynylene (—C:::C—, —C≡C—). Examples of alkynyl radicals include ethynyl, propynyl, hydroxypropynyl, butyn-1-yl, butyn-2-yl, pentyn-1-yl, pentyn-2-yl, 4-methoxypentyn-2-yl, 3-methylbutyn-1-yl, hexyn-1-yl, hexyn-2-yl, hexyn-3-yl, 3,3-dimethylbutyn-1-yl, and the like.

The term “amido,” as used herein, alone or in combination, refers to an amino group as described below attached to the parent molecular moiety through a carbonyl group. The term “C-amido” as used herein, alone or in combination, refers to a —C(═O)—NR₂ group with R as defined herein. The term “N-amido” as used herein, alone or in combination, refers to a RC(═O)NH— group, with R as defined herein.

The term “amino,” as used herein, alone or in combination, refers to —NRR′, wherein R and R′ are independently selected from the group consisting of hydrogen, alkenyl, alkoxy, alkoxyalkyl, alkoxycarbonyl, alkyl, alkylcarbonyl, aryl, arylalkenyl, arylalkyl, cycloalkyl, haloalkylcarbonyl, heteroaryl, heteroarylalkenyl, heteroarylalkyl, heterocycle, heterocycloalkenyl, and heterocycloalkyl, wherein the aryl, the aryl part of the arylalkenyl, the arylalkyl, the heteroaryl, the heteroaryl part of the heteroarylalkenyl and the heteroarylalkyl, the heterocycle, and the heterocycle part of the heterocycloalkenyl and the heterocycloalkyl can be optionally substituted as defined herein with one, two, three, four, or five substituents.

The term “aminoalkyl,” as used herein, alone or in combination, refers to an amino group attached to the parent molecular moiety through an alkyl group. Examples include aminomethyl, aminoethyl and aminobutyl.

The terms “aminocarbonyl” and “carbamoyl,” as used herein, alone or in combination, refer to an amino-substituted carbonyl group, wherein the amino group can be a primary or secondary amino group containing substituents selected from alkyl, aryl, aralkyl, cycloalkyl, cycloalkylalkyl radicals and the like.

The term “aminocarbonylalkyl,” as used herein, alone or in combination, refers to an aminocarbonyl radical attached to an alkyl radical, as described above. An example of such radicals is aminocarbonylmethyl. The term “amidino” denotes an —C(NH)NH₂ radical. The term “cyanoamidino” denotes an —C(N—CN)NH₂ radical.

The term “aralkenyl” or “arylalkenyl,” as used herein, alone or in combination, refers to an aryl group attached to the parent molecular moiety through an alkenyl group.

The term “aralkoxy” or “arylalkoxy,” as used herein, alone or in combination, refers to an aryl group attached to the parent molecular moiety through an alkoxy group.

The term “aralkyl” or “arylalkyl,” as used herein, alone or in combination, refers to an aryl group attached to the parent molecular moiety through an alkyl group.

The term “aralkylamino” or “arylalkylamino,” as used herein, alone or in combination, refers to an arylalkyl group attached to the parent molecular moiety through a nitrogen atom, wherein the nitrogen atom is substituted with hydrogen.

The term “aralkylidene” or “arylalkylidene,” as used herein, alone or in combination, refers to an aryl group attached to the parent molecular moiety through an alkylidene group

The term “aralkylthio” or “arylalkylthio,” as used herein, alone or in combination, refers to an arylalkyl group attached to the parent molecular moiety through a sulfur atom.

The term “aralkynyl” or “arylalkynyl,” as used herein, alone or in combination, refers to an aryl group attached to the parent molecular moiety through an alkynyl group.

The term “aralkoxycarbonyl,” as used herein, alone or in combination, refers to a radical of the formula aralkyl-O—C(O)— in which the term “aralkyl,” has the significance given above. Examples of an aralkoxycarbonyl radical are benzyloxycarbonyl (Z or Cbz) and 4-methoxyphenylmethoxycarbonyl (MOS).

The term “aralkanoyl,” as used herein, alone or in combination, refers to an acyl radical derived from an aryl-substituted alkanecarboxylic acid such as benzoyl, phenylacetyl, 3-phenylpropionyl (hydrocinnamoyl), 4-phenylbutyryl, (2-naphthyl)acetyl, 4-chlorohydrocinnamoyl, 4-aminohydrocinnamoyl, 4-methoxyhydrocinnamoyl, and the like. The term “aroyl” refers to an acyl radical derived from an arylcarboxylic acid, “aryl” having the meaning given below. Examples of such aroyl radicals include substituted and unsubstituted benzoyl or napthoyl such as benzoyl, 4-chlorobenzoyl, 4-carboxybenzoyl, 4-(benzyloxycarbonyl)benzoyl, 1-naphthoyl, 2-naphthoyl, 6-carboxy-2-naphthoyl, 6-(benzyloxycarbonyl)-2-naphthoyl, 3-benzyloxy-2-naphthoyl, 3-hydroxy-2-naphthoyl, 3-(benzyloxyformamido)-2-naphthoyl, and the like.

The term “aryl,” as used herein, alone or in combination, means a carbocyclic aromatic system containing one, two or three rings wherein such rings may be attached together in a pendent manner or may be fused. The term “aryl” embraces aromatic radicals such as benzyl, phenyl, naphthyl, anthracenyl, phenanthryl, indanyl, indenyl, annulenyl, azulenyl, tetrahydronaphthyl, and biphenyl.

The term “arylamino” as used herein, alone or in combination, refers to an aryl group attached to the parent moiety through an amino group, such as methylamino, N-phenylamino, and the like.

The terms “arylcarbonyl” and “aroyl,” as used herein, alone or in combination, refer to an aryl group attached to the parent molecular moiety through a carbonyl group.

The term “aryloxy,” as used herein, alone or in combination, refers to an aryl group attached to the parent molecular moiety through an oxygen atom.

The term “arylsulfonyl,” as used herein, alone or in combination, refers to an aryl group attached to the parent molecular moiety through a sulfonyl group.

The term “arylthio,” as used herein, alone or in combination, refers to an aryl group attached to the parent molecular moiety through a sulfur atom.

The terms “carboxy” or “carboxyl”, whether used alone or with other terms, such as “carboxyalkyl”, denotes —CO₂H.

The terms “benzo” and “benz,” as used herein, alone or in combination, refer to the divalent radical C₆H₄=derived from benzene. Examples include benzothiophene and benzimidazole.

The term “O-carbamyl” as used herein, alone or in combination, refers to a —OC(O)NRR′, group-with R and R′ as defined herein.

The term “N-carbamyl” as used herein, alone or in combination, refers to a ROC(O)NR′— group, with R and R′ as defined herein.

The term “carbonyl,” as used herein, when alone includes formyl [—C(O)H] and in combination is a —C(O)— group.

The term “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cyano,” as used herein, alone or in combination, refers to —CN.

The term “cycloalkyl,” as used herein, alone or in combination, refers to a saturated or partially saturated monocyclic, bicyclic or tricyclic alkyl radical wherein each cyclic moiety contains from 3 to 12, preferably five to seven, carbon atom ring members and which may optionally be a benzo fused ring system which is optionally substituted as defined herein. Examples of such cycloalkyl radicals include cyclopropyl, cyclobutyl, cyclopentyl, cyclohexyl, cycloheptyl, octahydronaphthyl, 2,3-dihydro-1H-indenyl, adamantyl and the like. “Bicyclic” and “tricyclic” as used herein are intended to include both fused ring systems, such as decahydronapthalene, octahydronapthalene as well as the multicyclic (multicentered) saturated or partially unsaturated type. The latter type of isomer is exemplified in general by bicyclo[2,2,2]octane, bicyclo[2,2,2]octane, bicyclo[1,1,1]pentane, camphor and bicyclo[3,2,1]octane.

The term “ester,” as used herein, alone or in combination, refers to a carboxyl group bridging two moieties linked at carbon atoms.

The term “ether,” as used herein, alone or in combination, refers to an oxy group bridging two moieties linked at carbon atoms.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a halohydrocarbyl group attached at two or more positions. Examples include fluoromethylene (—CFH—), difluoromethylene (—CF₂—), chloromethylene (—CHCl—) and the like.

The term “heteroalkyl,” as used herein, alone or in combination, refers to a stable straight or branched chain, or cyclic hydrocarbon radical, or combinations thereof, fully saturated or containing from 1 to 3 degrees of unsaturation, consisting of the stated number of carbon atoms and from one to three heteroatoms selected from the group consisting of O, N, and S, and wherein the nitrogen and sulfur atoms may optionally be oxidized and the nitrogen heteroatom may optionally be quaternized. The heteroatom(s) O, N and S may be placed at any interior position of the heteroalkyl group. Up to two heteroatoms may be consecutive, such as, for example, —CH₂—NH—OCH₃.

The term “heteroaryl,” as used herein, alone or in combination, refers to 3 to 7 membered, preferably 5 to 7 membered, unsaturated heterocyclic rings wherein at least one atom is selected from the group consisting of O, S, and N. Heteroaryl groups are exemplified by: unsaturated 3 to 7 membered heteromonocyclic groups containing 1 to 4 nitrogen atoms, for example, pyrrolyl, pyrrolinyl, imidazolyl, pyrazolyl, pyridyl, pyrimidinyl, pyrazinyl, pyridazinyl, triazolyl [e.g., 4H-1,2,4-triazolyl, 1H-1,2,3-triazolyl, 2H-1,2,3-triazolyl, etc.]tetrazolyl [e.g. 1H-tetrazolyl, 2H-tetrazolyl, etc.], etc.; unsaturated condensed heterocyclic group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl [e.g., tetrazolo[1,5-b]pyridazinyl, etc.], etc.; unsaturated 3 to 6-membered heteromonocyclic groups containing an oxygen atom, for example, pyranyl, furyl, etc.; unsaturated 3 to 6-membered heteromonocyclic groups containing a sulfur atom, for example, thienyl, etc.; unsaturated 3- to 6-membered heteromonocyclic groups containing 1 to 2 oxygen atoms and 1 to 3 nitrogen atoms, for example, oxazolyl, isoxazolyl, oxadiazolyl [e.g., 1,2,4-oxadiazolyl, 1,3,4-oxadiazolyl, 1,2,5-oxadiazolyl, etc.]etc.; unsaturated condensed heterocyclic groups containing 1 to 2 oxygen atoms and 1 to 3 nitrogen atoms [e.g. benzoxazolyl, benzoxadiazolyl, etc.]; unsaturated 3 to 6-membered heteromonocyclic groups containing 1 to 2 sulfur atoms and 1 to 3 nitrogen atoms, for example, thiazolyl, thiadiazolyl [e.g., 1,2,4-thiadiazolyl, 1,3,4-thiadiazolyl, 1,2,5-thiadiazolyl, etc.]and isothiazolyl; unsaturated condensed heterocyclic groups containing 1 to 2 sulfur atoms and 1 to 3 nitrogen atoms [e.g., benzothiazolyl, benzothiadiazolyl, etc.]and the like. The term also embraces radicals where heterocyclic radicals are fused with aryl radicals. Examples of such fused bicyclic radicals include benzofuryl, benzothienyl, and the like.

The term “heteroaralkenyl” or “heteroarylalkenyl,” as used herein, alone or in combination, refers to a heteroaryl group attached to the parent molecular moiety through an alkenyl group.

The term “heteroaralkoxy” or “heteroarylalkoxy,” as used herein, alone or in combination, refers to a heteroaryl group attached to the parent molecular moiety through an alkoxy group.

The term “heteroarylalkyl,” as used herein, alone or in combination, refers to a heteroaryl group attached to the parent molecular moiety through an alkyl group.

The term “heteroaralkylidene” or “heteroarylalkylidene,” as used herein, alone or in combination, refers to a heteroaryl group attached to the parent molecular moiety through an alkylidene group.

The term “heteroaryloxy,” as used herein, alone or in combination, refers to a heteroaryl group attached to the parent molecular moiety through an oxygen atom.

The term “heteroarylsulfonyl,” as used herein, alone or in combination, refers to a heteroaryl group attached to the parent molecular moiety through a sulfonyl group.

The terms “heterocycloalkyl” and, interchangeably, “heterocycle,” as used herein, alone or in combination, each refer to a saturated, partially unsaturated, or fully unsaturated monocyclic, bicyclic, or tricyclic heterocyclic radical containing at least one, preferably 1 to 4, and more preferably 1 to 2 heteroatoms as ring members, wherein each said heteroatom may be independently selected from the group consisting of nitrogen, oxygen, and sulfur, and wherein there are preferably 3 to 8 ring members in each ring, more preferably 3 to 7 ring members in each ring, and most preferably 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Heterocycle groups of the invention are exemplified by aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihy-dropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited.

The term “heterocycloalkylalkenyl,” as used herein, alone or in combination, refers to a heterocycle group attached to the parent molecular moiety through an alkenyl group.

The term “heterocycloalkylalkoxy,” as used herein, alone or in combination, refers to a heterocycle group attached to the parent molecular group through an oxygen atom.

The term “heterocycloalkylalkylidene,” as used herein, alone or in combination, refers to a heterocycle group attached to the parent molecular moiety through an alkylidene group.

The term “hydrazinyl” as used herein, alone or in combination, refers to two amino groups joined by a single bond, i.e., —N—N—.

The term “hydroxy,” as used herein, alone or in combination, refers to —OH.

The term “hydroxyalkyl,” as used herein, alone or in combination, refers to a hydroxy group attached to the parent molecular moiety through an alkyl group.

The term “imino,” as used herein, alone or in combination, refers to ═N—.

The term “iminohydroxy,” as used herein, alone or in combination, refers to ═N(OH) and ═N—O—.

The phrase “in the main chain” refers to the longest contiguous or adjacent chain of carbon atoms starting at the point of attachment of a group to the compounds of this invention.

The term “isocyanato” refers to a —NCO group.

The term “isothiocyanato” refers to a —NCS group.

The phrase “linear chain of atoms” refers to the longest straight chain of atoms independently selected from carbon, nitrogen, oxygen and sulfur.

The term “lower,” as used herein, alone or in combination, means containing from 1 to and including 6 carbon atoms.

The term “mercaptoalkyl” as used herein, alone or in combination, refers to an R′SR— group, where R and R′ are as defined herein.

The term “mercaptomercaptyl” as used herein, alone or in combination, refers to a RSR′S— group, where R and R′ are as defined herein.

The term “mercaptyl” as used herein, alone or in combination, refers to an RS— group, where R is as defined herein.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The terms “sulfonate,” “sulfonic acid,” and “sulfonic,” as used herein, alone or in combination, refer the —SO₃H group and its anion as the sulfonic acid is used in salt formation.

The term “sulfanyl,” as used herein, alone or in combination, refers to —S—.

The term “sulfinyl,” as used herein, alone or in combination, refers to —S(O)—.

The term “sulfonyl,” as used herein, alone or in combination, refers to —SO₂—.

The term “N-sulfonamido” refers to a RS(═O)₂NR′— group with R and R′ as defined herein.

The term “S-sulfonamido” refers to a —S(═O)₂NRR′, group, with R and R′ as defined herein.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

The term “thiol,” as used herein, alone or in combination, refers to an —SH group.

The term “thiocarbonyl,” as used herein, when alone includes thioformyl —C(S)H and in combination is a —C(S)— group.

The term “N-thiocarbamyl” refers to an ROC(S)NR′— group, with R and R′ as defined herein.

The term “O-thiocarbamyl” refers to a —OC(S)NRR′, group with R and R′ as defined herein.

The term “thiocyanato” refers to a —CNS group.

The term “trihalomethanesulfonamido” refers to a X₃CS(O)₂NR— group with X is a halogen and R as defined herein.

The term “trihalomethanesulfonyl” refers to a X₃CS(O)₂— group where X is a halogen.

The term “trihalomethoxy” refers to a X₃CO— group where X is a halogen.

The term “trisubstituted silyl,” as used herein, alone or in combination, refers to a silicone group substituted at its three free valences with groups as listed herein under the definition of substituted amino. Examples include trimethysilyl, tert-butyldimethylsilyl, triphenylsilyl and the like.

When a group is defined to be “null,” what is meant is that said group is absent.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, arylthio, lower alkylsulfinyl, lower alkylsulfonyl, arylsulfinyl, arylsulfonyl, arylthio, sulfonate, sulfonic acid, trisubstituted silyl, N₃, NHCH₃, N(CH₃)₂, SH, SCH₃, C(O)CH₃, CO₂CH₃, CO₂H, C(O)NH₂, pyridinyl, thiophene, furanyl, lower carbamate, and lower urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

The term R or the term R′, appearing by itself and without a number designation, unless otherwise defined, refers to a moiety selected from the group consisting of hydrogen, alkyl, cycloalkyl, heteroalkyl, aryl, heteroaryl and heterocycloalkyl. Such R and R′ groups should be understood to be optionally substituted as defined herein. Whether an R group has a number designation or not, every R group, including R, R′ and Rⁿ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence.

Asymmetric centers exist in the compounds of the present invention. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds of the present invention may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

The term “bond” refers to a covalent linkage between two atoms, or two moieties when the atoms joined by the bond are considered to be part of larger substructure. A bond may be single, double, or triple unless otherwise specified.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

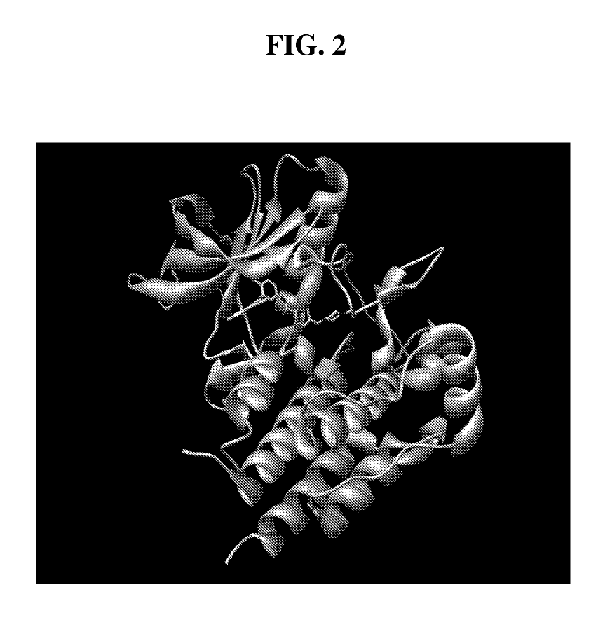

c-Kit is a protein that has: (1) enzymatic function as it is a kinase and carries out auto-phosphorylation; (2) binding function as it binds stem cell factor and other ligands by its extracellular domain; and (3) signaling function as a result of the enzymatic and binding functions. FIG. 2 is a three-dimensional depiction of the c-Kit protein.

The c-Kit protein is often designated as KIT in the literature together with a wide variety of other possible variations, including but not limited to, c-kit, kit, KIT, c-kit, c-Kit, and c-KIT. Likewise, the gene encoding c-Kit is often designated in the literature as kit or c-kit. Moreover, as with protein designations, the terms c-kit, c-Kit, c-KIT, KIT, KIT, and c-Kit can be associated with the gene that encodes the protein and variations thereof. Therefore, as used herein, any one of a number of possible variation of the terms designating the c-Kit protein and the gene encoding the protein can and may be used interchangeably herein.

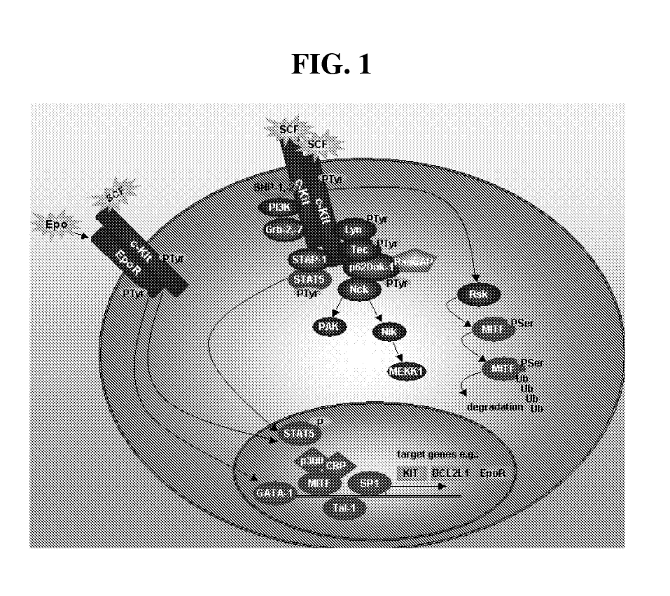

c-Kit is a type of receptor tyrosine kinase (RTK) involved in signal transduction as shown in the schematic of FIG. 1. In general, RTKs are monomeric surface receptors that dimerize upon activation. RTKs have an extracellular binding domain, a transmembrane domain, and an intracellular kinase domain. Ligand binding to the extracellular domain induces dimerization of the surface receptor which in turn induces phosphorylation of tyrosine residues within an “activation loop” of the intracellular kinase domain. c-Kit belongs to a family of RTK's termed the PDGF-Receptor family which includes PDGFRα, PDGFRβ, CSF1R, and FLK2.

c-Kit is specifically activated by its cognate ligand, stem cell factor (SCF). SCF is expressed by fibroblasts and endothelial cells throughout the body whereas c-Kit expression is more restricted and predominantly found on primitive hematopoietic cells, mast cells, melanocytes, testis, brain, vascular endothelial cells, interstitial cells of Cajal, breast glandular epithelial cells and sweat glands.

The phrase “therapeutically effective” is intended to qualify the amount of active ingredients used in the treatment of a disease or disorder. This amount will achieve the goal of reducing or eliminating the said disease or disorder.

As used herein, reference to “treatment” of a patient is intended to include prophylaxis. The term “patient” means all mammals including humans. Examples of patients include humans, cows, dogs, cats, goats, sheep, pigs, and rabbits. Preferably, the patient is a human.

The term “prodrug” refers to a compound that is made more active in vivo. The present compounds can also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound. The term “therapeutically acceptable prodrug,” refers to those prodrugs or zwitterions which are suitable for use in contact with the tissues of patients without undue toxicity, irritation, and allergic response, are commensurate with a reasonable benefit/risk ratio, and are effective for their intended use.

The term “therapeutically acceptable salt,” as used herein, represents salts or zwitterionic forms of the compounds of the present invention which are water or oil-soluble or dispersible; which are suitable for treatment of diseases without undue toxicity, irritation, and allergic-response; which are commensurate with a reasonable benefit/risk ratio; and which are effective for their intended use. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds of the present invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds of the compounds of the present invention and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

The compounds of the present invention can exist as therapeutically acceptable salts. The present invention includes compounds listed above in the form of salts, in particular acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. For a more complete discussion of the preparation and selection of salts, refer to Pharmaceutical Salts: Properties, Selection, and Use (Stahl, P. Heinrich. Wiley-VCHA, Zurich, Switzerland, 2002).

Thus, preferred salts include hydrochloride, hydrobromide, sulfonate, citrate, tartrate, phosphonate, lactate, pyruvate, acetate, succinate, oxalate, fumarate, malate, oxaloacetate, methanesulfonate, ethanesulfonate, p-toluenesulfonate, benzenesulfonate and isethionate salts of compounds of the present invention. A salt of a compound can be made by reacting the appropriate compound in the form of the free base with the appropriate acid.

While it may be possible for the compounds of the subject invention to be administered as the raw chemical, it is also possible to present them as a pharmaceutical formulation. Accordingly, the subject invention provides a pharmaceutical formulation comprising a compound or a pharmaceutically acceptable salt, ester, prodrug or solvate thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences. The pharmaceutical compositions of the present invention may be manufactured in a manner that is itself known, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. All methods include the step of bringing into association a compound of the subject invention or a pharmaceutically acceptable salt, ester, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Compounds of the present invention may be administered topically, that is by non-systemic administration. This includes the application of a compound of the present invention externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient may comprise, for topical administration, from 0.001% to 10% w/w, for instance from 1% to 2% by weight of the formulation. It may however comprise as much as 10% w/w but preferably will comprise less than 5% w/w, more preferably from 0.1% to 1% w/w of the formulation.

Gels for topical or transdermal administration of compounds of the subject invention may comprise, generally, a mixture of volatile solvents, nonvolatile solvents, and water. The volatile solvent component of the buffered solvent system may preferably include lower (C₁-C₆) alkyl alcohols, lower alkyl glycols and lower glycol polymers. More preferably, the volatile solvent is ethanol. The volatile solvent component is thought to act as a penetration enhancer, while also producing a cooling effect on the skin as it evaporates. The nonvolatile solvent portion of the buffered solvent system is selected from lower alkylene glycols and lower glycol polymers. Preferably, propylene glycol is used. The nonvolatile solvent slows the evaporation of the volatile solvent and reduces the vapor pressure of the buffered solvent system. The amount of this nonvolatile solvent component, as with the volatile solvent, is determined by the pharmaceutical compound or drug being used. When too little of the nonvolatile solvent is in the system, the pharmaceutical compound may crystallize due to evaporation of volatile solvent, while an excess will result in a lack of bioavailability due to poor release of drug from solvent mixture. The buffer component of the buffered solvent system may be selected from any buffer commonly used in the art; preferably, water is used. The preferred ratio of ingredients is about 20% of the nonvolatile solvent, about 40% of the volatile solvent, and about 40% water. There are several optional ingredients which can be added to the topical composition. These include, but are not limited to, chelators and gelling agents. Appropriate gelling agents can include, but are not limited to, semisynthetic cellulose derivatives (such as hydroxypropylmethylcellulose) and synthetic polymers, and cosmetic agents.

Lotions according to the present invention include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes according to the present invention are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy base. The base may comprise hydrocarbons such as hard, soft or liquid paraffin, glycerol, beeswax, a metallic soap; a mucilage; an oil of natural origin such as almond, corn, arachis, castor or olive oil; wool fat or its derivatives or a fatty acid such as steric or oleic acid together with an alcohol such as propylene glycol or a macrogel. The formulation may incorporate any suitable surface active agent such as an anionic, cationic or non-ionic surfactant such as a sorbitan ester or a polyoxyethylene derivative thereof. Suspending agents such as natural gums, cellulose derivatives or inorganic materials such as silicaceous silicas, and other ingredients such as lanolin, may also be included.

Drops according to the present invention may comprise sterile aqueous or oily solutions or suspensions and may be prepared by dissolving the active ingredient in a suitable aqueous solution of a bactericidal and/or fungicidal agent and/or any other suitable preservative, and preferably including a surface active agent. The resulting solution may then be clarified by filtration, transferred to a suitable container which is then sealed and sterilized by autoclaving or maintaining at 98-100° C. for half an hour. Alternatively, the solution may be sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Formulations for topical administration in the mouth, for example buccally or sublingually, include lozenges comprising the active ingredient in a flavored basis such as sucrose and acacia or tragacanth, and pastilles comprising the active ingredient in a basis such as gelatin and glycerin or sucrose and acacia.

For administration by inhalation the compounds according to the invention are conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

It should be understood that in addition to the ingredients particularly mentioned above, the formulations of this invention may include other agents conventional in the art having regard to the type of formulation in question, for example those suitable for oral administration may include flavoring agents.

The compounds of the invention may be administered orally or via injection at a dose of from 0.1 to 500 mg/kg per day. The dose range for adult humans is generally from 5 mg to 2 g/day. Tablets or other forms of presentation provided in discrete units may conveniently contain an amount of compound of the invention which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds of the subject invention can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In certain instances, it may be appropriate to administer at least one of the compounds described herein (or a pharmaceutically acceptable salt, ester, or prodrug thereof) in combination with another therapeutic agent. By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for diabetes involving administration of one of the compounds described herein, increased therapeutic benefit may result by also providing the patient with another therapeutic agent for diabetes. In any case, regardless of the disease, disorder or condition being treated, the overall benefit experienced by the patient may simply be additive of the two therapeutic agents or the patient may experience a synergistic benefit.

In any case, the multiple therapeutic agents (at least one of which is a compound of the present invention) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Thus, in another aspect, the present invention provides methods for treating c-Kit-mediated disorders in a human or animal subject in need of such treatment comprising administering to said subject an amount of a compound of the present invention effective to reduce or prevent said disorder in the subject in combination with at least one additional agent for the treatment of said disorder that is known in the art. In a related aspect, the present invention provides therapeutic compositions comprising at least one compound of the present invention in combination with one or more additional agents for the treatment of c-Kit-mediated disorders.

The compounds of the subject invention may be useful for the treatment or disorders of a wide variety of condition where inhibition or modulation of c-Kit is useful. Disorders or conditions advantageously treated by the compounds of the subject invention include the prevention or treatment of cancer, such as colorectal cancer, and cancer of the breast, lung, prostate, bladder, cervix and skin. Compounds of the invention may be used in the treatment and prevention of neoplasias including but not limited to brain cancer, bone cancer, a leukemia, a lymphoma, epithelial cell-derived neoplasia (epithelial carcinoma) such as basal cell carcinoma, adenocarcinoma, gastrointestinal cancer such as lip cancer, mouth cancer, esophogeal cancer, small bowel cancer and stomach cancer, colon cancer, liver cancer, bladder cancer, pancreas cancer, ovary cancer, cervical cancer, lung cancer, breast cancer and skin cancer, such as squamous cell and basal cell cancers, prostate cancer, renal cell carcinoma, and other known cancers that effect epithelial cells throughout the body. The neoplasia can be selected from gastrointestinal cancer, liver cancer, bladder cancer, pancreas cancer, ovary cancer, prostate cancer, cervical cancer, lung cancer, breast cancer and skin cancer, such as squamous cell and basal cell cancers. The present compounds and methods can also be used to treat the fibrosis which occurs with radiation therapy. The present compounds and methods can be used to treat subjects having adenomatous polyps, including those with familial adenomatous polyposis (FAP). Additionally, the present compounds and methods can be used to prevent polyps from forming in patients at risk of FAP.

Because a c-kit disorder or condition results from inappropriate protein kinase activity, in particular, activity of protein kinases having c-Kit intracellular domain, particularly c-Kit ATP-binding site, or c-Kit activation loop, this type of condition may include, but is not limited to disorders of hematopoietic cells, mast cells, melanocytes, testis, brain, vascular endothelial cells, interstitial cells of Cajal, breast glandular epithelial cells or sweat glands.

Specific treatable neoplasms include systemic mast cell disorders, seminoma, acute myelogenous leukemia (AML), gastrointestinal stromal tumors (GISTs) or hypopigmentary disorders.

A c-Kit disorder or condition may be due to a mutation in c-Kit in a codon that encodes amino acids that form the enzyme pocket of c-Kit, such as Asp816Val, that alters the activation loop. Wild type c-Kit contains an aspartic acid at position 816 of the activation loop. A c-Kit protein having a valine at position 816 instead of aspartic acid is a mutant c-Kit. The Asp816Val mutation appears to stabilize an active conformation of the activation loop domain of the enzyme pocket resulting in oncogenic kinase activation. Mutated kinases are constitutive, i.e., they are always activated. Activation of c-Kit involves receptor dimerization and, in mutated c-Kit, is ligand-independent and results in oncogenic activation of the kinase receptor. The KIT-D816 mutation confers a poor prognosis to AML1-ETO-positive AML and antagonists as provided herein are useful for treatment, prevention, screening and diagnosis.

A c-Kit condition or disorder can be the result of a regulatory-type mutation that affects regions other than the enzyme pocket. Protein residues may be mutated in a membrane domain or a juxtamembrane domain that normally inhibits ligand-independent kinase activation.

In addition, c-Kit disorder may be due to over-expression, inappropriate timing of activation, or by inappropriate levels or activity of ligands that bind to the kinase receptor.

The compounds of the such invention may be also used in as combination therapy together with existing tyrosine kinase inhibitors such as dasatinib (BMS-354825, Bristol Myers Squibb; a small-molecule, ATP-competitive inhibitor of SRC and ABL tyrosine kinases having a potency in the low nanomolar range), imatinib mesylate (a.k.a. IMATINIB®, STI-571, GLEEVEC® or GLIVEC®, Novartis), gefitinib (IRESSA®, Astra Zeneca), erlotinib (TARCEVA®, OSI Pharmaceuticals), AMN107 (Novartis), and sunitinib malate (a.k.a. SU11248, SUTENT®, Pfizer). c-Kit mutations known to be sensitive to IMATINIB®, include Val560Gly, Glu839Lys, and Asp820Gly. c-Kit mutations known to be resistant to IMATINIB®, include Asp816Val/Phe/Tyr.

Other disorders or conditions which can be advantageously treated by the compounds of the present invention are inflammation. The compounds of the present invention are useful as anti-inflammatory agents with the additional benefit of having significantly less harmful side effects. The compounds are useful to treat arthritis, including but not limited to rheumatoid arthritis, spondyloarthropathies, gouty arthritis, osteoarthritis, systemic lupus erythematosus, juvenile arthritis, acute rheumatic arthritis, enteropathic arthritis, neuropathic arthritis, psoriatic arthritis, and pyogenic arthritis. The compounds are also useful in treating osteoporosis and other related bone disorders. These compounds can also be used to treat gastrointestinal conditions such as inflammatory bowel disease, Crohn's disease, gastritis, irritable bowel syndrome and ulcerative colitis. The compounds may also be used in the treatment of pulmonary inflammation, such as that associated with viral infections and cystic fibrosis. In addition, compounds of invention are also useful in organ transplant patients either alone or in combination with conventional immunomodulators.

The present compounds may also be used in co-therapies, partially or completely, in place of other conventional anti-inflammatory therapies, such as together with steroids, NSAIDs, COX-2 selective inhibitors, 5-lipoxygenase inhibitors, LTB₄ antagonists and LTA₄ hydrolase inhibitors. The compounds of the subject invention may also be used to prevent tissue damage when therapeutically combined with antibacterial or antiviral agents.

Besides being useful for human treatment, these compounds are also useful for veterinary treatment of companion animals, exotic animals and farm animals, including mammals, rodents, and the like. More preferred animals include horses, dogs, and cats.

Besides being useful for human treatment, the compounds and formulations of the present invention are also useful for veterinary treatment of companion animals, exotic animals and farm animals, including mammals, rodents, and the like. More preferred animals include horses, dogs, and cats.

All references, patents or applications, U.S. or foreign, cited in the application are hereby incorporated by reference as if written herein.

The compounds of the invention may be administered orally or via injection at a dose of from 0.1 to 500 mg/kg per day. The dose range for adult humans is generally from 5 mg to 2 g/day. Tablets or other forms of presentation provided in discrete units may conveniently contain an amount of compound of the invention which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds of the subject invention can be administered orally or by injection (intravenous or subcutaneous). The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

### General Synthetic Methods for Preparing Compounds

The following scheme can be used to practice the present invention.

Examples 1-56 can be synthesized using the following general synthetic procedure set forth in Scheme 1.

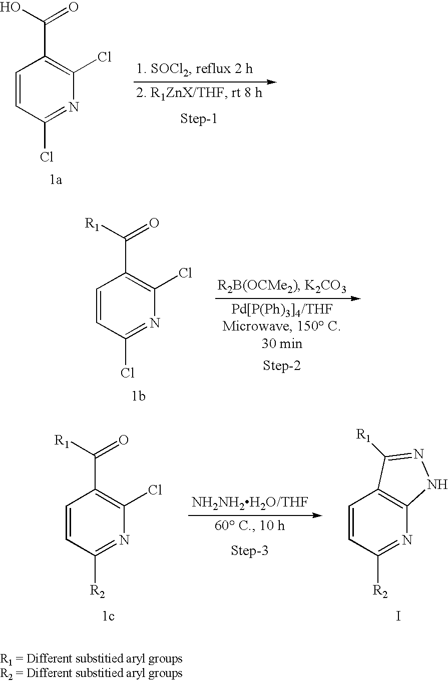

All chemicals and solvents were obtained from Sigma-Aldrich (Milwaukee, Wis.) or Fisher Scientific (Pittsburgh, Pa.) and used without further purification. ¹H-NMR and ¹³C-NMR spectra were recorded on an IBM-Brucker Avance 300 (300 MHz for ¹H-NMR and 75.48 MHz for ¹³C-NMR), and IBM-Brucker Avance 500 (500 MHz for ¹H-NMR and 125.76 MHz for ¹³C-NMR), spectrometers. Chemical shifts (δ) are determined relative to CDCl₃ (referenced to 7.27 ppm (δ) for ¹H-NMR and 77.0 ppm for ¹³C-NMR) or DMSO-d₆ (referenced to 2.49 ppm (δ) for ¹H-NMR and 39.5 ppm for ¹³C-NMR). Proton-proton coupling constants (J) are given in Hertz and spectral splitting patterns are designated as singlet (s), doublet (d), triplet (t), quadruplet (q), multiplet or overlapped (m), and broad (br). Low resolution mass spectra (ionspray, a variation of electrospray) were acquired on a Perkin-Elmer Sciex API 100 spectrometer or Applied Biosystems Q-trap 2000 LC-MS-MS. Flash chromatography was performed using Merk silica gel 60 (mesh size 230-400 ASTM) or using an Isco (Lincon, Nebr.) combiFlash Companion or SQ16x flash chromatography system with RediSep columns (normal phase silica gel (mesh size 230-400ASTM) and Fisher Optima™ grade solvents. Thin-layer chromatography (TLC) was performed on E. Merk (Darmstadt, Germany) silica gel F-254 aluminum-backed plates with visualization under UV (254 nm) and by staining with potassium permanganate or ceric ammonium molybdate.

Structural or chemical diversity was first introduced at the beginning of the synthesis with the condensation of 2,6-dichloro nicotinic acid chloride (1a of Scheme 1) (PCT International Patent Application published as WO 2005/073217 to SmithKline Beecham, Appl'n No. PCT/GB2005/000266 having an international filing date of Jan. 27, 2005; Quiroga, et al., (2001) Journal of Heterocyclic Chemistry 38, 53-60.) with various R1-zinc halides (e.g., bromide or iodide) in presence of tetrakis(triphenylphosphine)palladium in THF to give 1b of Scheme 1.

A second round of structural or chemical diversity was introduced by the condensation of 1b with various R₂-boronate esters [R₂B(OR)₂] by means of a Suzuki coupling (Sun, et al., U.S. Published Patent Application No. 2005/0009832, filed May 14, 2004) using microwave conditions at 150° C. for 30 min giving 1c. Subsequent treatment with hydrazine hydrate gave the compounds of Formula I.

Illustrative of Scheme 1 is the synthesis of 3-{(4-Methyloxyphenyl)-1H-pyrazolo[4,3-b]pyridin-6-yl}acetanilide.

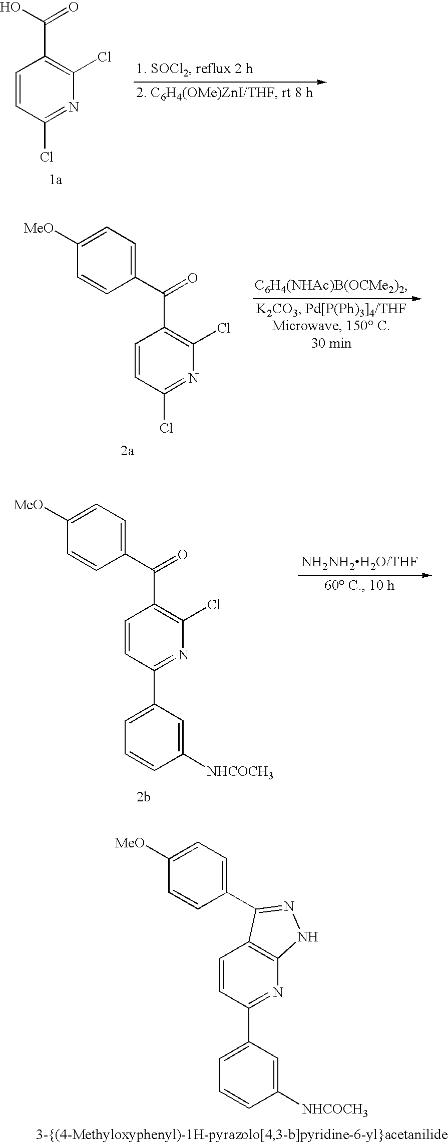

### Step 1

### Preparation of compound 2a

### (2-6-Dichloro-3-pyridinyl)(4-methyloxyphenyl)methanone

4,6-Dichloro-3-pyridine carboxylic acid (2,6-Dichloronicotinic acid) 1a (2.5 g, 13.02 mmol) and thionyl chloride (15 mL) were heated at 105° C. for 5 h. Excess thionyl chloride was removed under vacuum and the residual oil was dissolved in dry THF (35 mL). Tetrakis(triphenylphosphine)palladium (140 mg) and 4-methoxyphenylzinc iodide (0.5M, 30 mL) were added to the solution which was stirred at room temperature for 10 h. The reaction mixture was treated with saturated ammonium chloride solution, volatile solvent was removed in vacuum and then extracted with ethyl acetate (100 mL×2). The combined organic phase was washed with brine, dried (Na₂SO₄) and concentrated. The crude product was purified by flash column chromatography over silica gel, using polarity gradient 5-20% EtOAc in hexane to yield ketone 2a (3.05 g 81%) as a reddish solid; ¹H NMR (600 MHz, CDCl₃) 7.76 (d, 2H, J=9.0 Hz), 7.69 (d, 1H, J=7.8 Hz), 7.40 (d, 1H, J=7.8 Hz), 6.96 (d, 1H, J=9.0 Hz), 3.90 (s, 3H); ¹³C NMR δ 190.9, 164.7, 151.1, 147.1 140.0, 133.9, 132.5, 128.5, 123.0, 114.3, 55.7; MS (C₁₃H₉Cl₂NO₂) calcd. 281.002 found 282.5.

### Step 2

### Preparation of compound 2b

### (2-6-Dichloro-3-pyridinyl)(4-methyloxyphenyl)methanone

A mixture of (2-6-dichloro-3-pyridinyl)(4-methyloxyphenyl)methanone (300 mg, 1.06 mmol), 3′-(4,4,5,5-tetramethyl-1,3,2-dioxaborolan-2-yl)acetanilide, potassium carbonate (200 mg), tetrakis(triphenylphosphine)palladium (80 mg) in THF (4 mL) was flashed with argon and then subjected to microwave at 155° C. for 30 min. THF was removed and absorbed onto silica, then purified by flash column chromatography over silica gel, using polarity gradient 5-50% EtOAc in hexane to yield acetanilide derivative 3 (200 mg 49%) as a yellow oil; ¹H NMR (600 MHz DMSO-d₆) 10.17 (s, 1H), 8.32 (s, 1H), 8.08 (s, 2H), 7.81 (m, 4H), 7.47 (t, 1H, J=7.8 Hz), 7.10 (d, 2H, J=9.0 Hz), 3.87 (s, 3H), 2.08 (s, 3H); ¹³C NMR δ 191.3, 168.5, 164.2, 157.4, 145.9, 140.1, 139.5, 136.8, 133.1, 132.3, 129.5, 128.4, 121.5, 120.8, 119.2, 117.3, 114.5, 55.7, 24.0; MS (C₂₁H₁₇ClN₂O₃) calcd. 380.093 found 381.3.

### Step 3

### Preparation of 3-{(4-Methyloxyphenyl)-1H-pyrazolo[4,3-b]pyridin-6-yl}acetanilide

3-[6-chloro-5-{(4-methyloxyphenyl)carbonyl}-2-pyridinyl]acetanilide (85 mg, 0.22 mmol) and hydrazine hydrate (0.7 mL) were mixed in THF (1.5 mL) and heated for 10 h. The reaction mixture was cooled and THF was removed in vacuum. CHCl₃ (2 ml) was added and shaken well. The solid product was filtered, washed with chloroform (3×3 mL) and dried. The residue was triturated with ethyl acetate to give this compound as a yellow solid (45 mg, 53%).

The invention is further illustrated by the following examples.

### Example 1

### 3-{(4-Methyloxyphenyl)-1H-pyrazolo[4,3-b]pyridin-6-yl}acetanilide

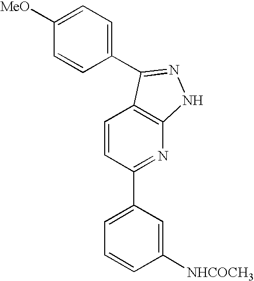

¹H NMR (600 MHz, DMSO-d₆) 13.71 (bs, 1H), 10.15 (s, 1H), 8.60 (d, 1H, J=8.4 Hz), 8.46 (s, 1H), 7.99 (d, 2H, J=8.4 Hz), 7.82 (d, 1H, J=8.4 Hz), 7.76 (d, 2H, J=8.4 Hz), 7.72 (d, 2H, J=7.8 Hz), 7.46 (t, 1H, J=7.8 Hz), 7.10 (d, 2H, J=8.4 Hz), 3.84 (s, 3H), 2.11 (s, 3H); ¹³C NMR δ 168.5, 159.3, 155.2, 153.1, 142.5, 139.9, 139.1, 131.3, 129.2, 127.7, 125.7, 121.9, 120.0, 117.7, 114.4, 114.3, 110.9, 55.2, 24.1; MS (C₂₁H₁₈N₄O₂) calcd. 358.143 found 359.5.

### Example 2

### 3-[3-(4-Fluoro-3-methylphenyl)-1H-pyrazolo[3,4-b]pyridin-6-yl]acetanilide

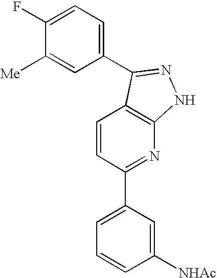

¹H NMR (600 MHz, DMSO-d₆) δ 13.93 (s, 1H), 10.18 (s, 1H), 8.67 (d, 1H, J=9.0 Hz), 8.47 (s, 1H), 7.78 (m, 5H), 7.45 (m, 2H), 2.32 (s, 3H), 2.11 (s, 3H); ¹³C NMR δ 168.9, 162.3, 160.7, 155.9, 153.6, 141.9, 140.4, 139.5, 133.4, 132.7, 131.7, 129.7, 124.6, 122.6, 122.4, 120.6, 118.2, 118.2, 115.2, 113.0, 112.8, 111.5, 24.5, 14.5; MS (C₂₁H₁₇FN₄O) calcd. 360.138 found 361.6.

### Example 3

### 3-[3-(4-Fluorophenyl)-1H-pyrazolo[3,4-b]pyridin-6-yl]acetanilide

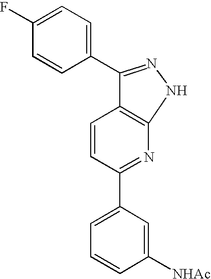

¹H NMR (600 MHz, DMSO-d₆) δ 13.89 (s, 1H), 10.15 (s, 1H), 8.64 (d, 1H, J=8.4 Hz), 8.47 (s, 1H), 8.81 (m, 2H), 7.80 (m, 3H), 7.47 (m, 1H), 7.38 (m, 2H), 2.11 (s, 3H); ¹³C NMR δ 168.5, 162.8, 161.2, 155.4, 153.1, 141.7, 139.9, 139.0, 131.2, 129.7, 129.2, 128.5, 128.4, 121.9, 120.1, 117.7, 115.9, 115.8, 114.6, 110.9, 24.0; MS (C₂₀H₁₅FN₄O) calcd. 346.123 found 347.4.

### Example 4

### 3-[3-(4-Chloro-3-fluorophenyl)-1H-pyrazolo[3,4-b]pyridin-6-yl]acetanilide

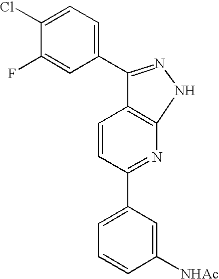

¹H NMR (600 MHz, DMSO-d₆) δ 13.89 (s, 1H), 10.17 (s, 1H), 8.72 (d, 1H, J=9.0 Hz), 8.46 (s, 1H), 8.04 (dd, 1H, J=10.8, 1.8 Hz), 7.95 (dd, 1H, J=8.4, 1.8 Hz), 7.83 (m, 2H), 7.75 (t, 1H, J=8.4 Hz), 7.71 (d, 1H, J=7.8 Hz), 7.47 (t, 1H, J=7.8 Hz), 2.10 (s, 3H); ¹³C NMR δ 169.0, 158.9, 157.3, 156.1, 153.6, 140.9, 140.4, 139.4, 134.6, 131.6, 129.7, 123.9, 122.4, 120.7, 119.6, 118.2, 115.4, 114.8, 114.6, 111.4, 24.5; MS (C₂₀H₁₄ClFN₄O) calcd. 380.084 found 381.3.

### Example 5

### 3-[3-(4-Ethoxycarbonyl)-1H-pyrazolo[3,4-b]pyridin-6-yl]acetanilide

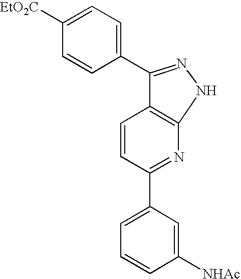

¹H NMR (600 MHz, DMSO-d₆) δ 13.71 (bs, 1H), 10.24 (s, 1H), 8.71 (d, 1H, J=8.4 Hz), 8.49 (s, 1H), 8.23 (d, 2H, J=8.4 Hz), 8.12 (d, 2H, J=8.4 Hz), 7.83 (d, 2H, J=8.4 Hz), 7.72 (d, 1H, J=7.8 Hz), 7.46 (t, 1H, J=7.8 Hz), 4.36 ((q, 2H, J=7.2 Hz)), 1.36 (t, 3H, J=7.2 Hz); ¹³C NMR δ 168.5, 165.4, 155.6, 153.2, 141.4, 139.9, 138.9, 137.5, 131.2, 129.8, 129.2, 129.1, 126.4, 121.9, 120.2, 117.7, 114.4, 115.0, 111.2, 60.8, 24.0, 14.2; MS (C₂₃H₂₀N₄O₃) calcd. 400.1535 found 401.4.

### Example 6

### 4-[3-(4-Ethoxycarbonyl)-1H-pyrazolo[3,4-b]pyridin-6-yl]-2,6-dimethyl]phenol

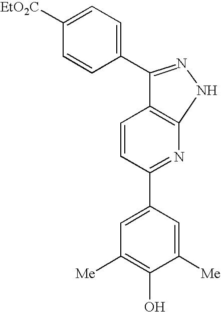

¹H NMR (600 MHz, DMSO-d₆) δ 13.71 (bs, 1H), 8.58 (d, 1H, J=9.0 Hz), 8.21 (d, 2H, J=6.6 Hz), 8.11 (d, 2H, J=6.6 Hz), 7.83 (s, 2H), 7.81 (d, 2H, J=9.0 Hz), 4.36 (q, 2H, J=7.2 Hz), 1.36 (t, 3H, J=7.2 Hz); ¹³C NMR δ 168.9, 156.5, 155.6, 153.8, 141.8, 138.2, 131.2, 130.3, 129.6, 129.4, 127.8, 126.4, 124.9, 114.8, 110.8, 110.8, 61.2, 17.3, 14.7; MS (C₂₃H₂₁N₃O₃) calcd. 387.1583 found 388.4.

### Example 7

### [4-{3-(4-Chloro-3-fluorophenyl)-1H-pyrazolo[3,4-b]pyridin-6-yl}-2,6-dimethyl]phenol

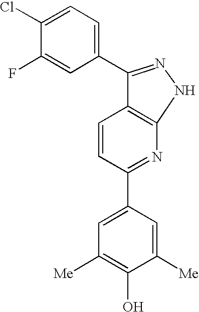

¹H NMR (600 MHz, DMSO-d₆) δ 13.71 (bs, 1H), 8.56 (d, 1H, J=9.0 Hz), 7.99 (dd, 1H, J=10.8, 1.8 Hz), 7.91 (dd, 1H, J=10.8, 1.8 Hz), 7.81 (s, 2H), 7.77 (d, 2H, J=9.0 Hz), 7.70 (t, 1H, J=8.4 Hz), 2.28 (s, 6H); ¹³C NMR δ 158.8, 157.2, 156.5, 155.6, 153.8, 140.8, 134.9, 131.6, 131.1, 129.6, 127.8, 124.9, 123.9, 119.3, 114.7, 114.5, 110.5, 17.3; MS (C₂₀H₁₅ClFN₃O₃) calcd. 367.0888 found 368.3.

### Example 8

### 1-[3-(4-Ethoxycarbonyl)-1H-pyrazolo[3,4-b]pyridin-6-yl]-3,5-dimethoxy]benzene

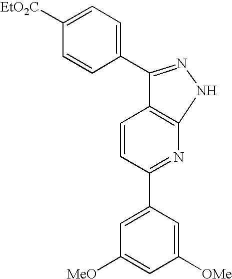

¹H NMR (600 MHz, DMSO-d₆) δ 14.03 (bs, 1H), 8.68 (d, 1H, J=9.0 Hz), 8.23 (d, 2H, J=8.4 Hz), 8.11 (d, 2H, J=8.4 Hz), 7.93 (d, 1H, J=9.0 Hz), 7.36 (d, 2H, J=2.4 Hz), 6.65 (s, 2H), 4.36 (q, 2H, J=7.2 Hz), 3.86 (s, 6H), 1.36 (t, 1H, J=7.2 Hz); ¹³C NMR δ 165.4, 160.8, 155.3, 153.1, 141.4, 140.5, 137.5, 131.1, 129.8, 129.1, 126.4, 115.3, 111.4, 105.1, 101.6, 60.8, 55.4, 14.2; MS (C₂₃H₂₁N₃O₄) calcd. 403.1532 found 404.4.

### Example 9

### 1-[3-(4-fluoro-3-methyl)-1H-pyrazolo[3,4-b]pyridin-6-yl]-3,5-dimethoxy]benzene

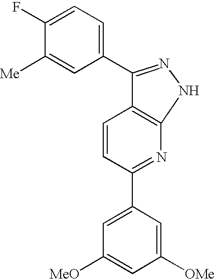

¹H NMR (600 MHz, DMSO-d₆) δ 14.03 (bs, 1H), 8.62 (d, 1H, J=8.4 Hz), 7.87 (d, 2H, J=8.4 Hz), 7.80 (d, 1H, J=7.8 Hz), 7.75 (d, 1H, J=11.4 Hz), 7.44 (t, 1H, J=7.8 Hz), 7.34 (d, 2H, J=1.8 Hz), 6.64 (t, 1H, J=1.8 Hz), 3.86 (s, 6H), 2.31 (s, 3H); ¹³C NMR δ 161.8, 160.8, 160.2, 155.1, 153.0, 141.4, 140.6, 132.8, 132.2, 131.1, 124.1, 122.2, 115.0, 122.5, 122.3, 111.1, 105.1, 101.6, 55.4, 14.0; MS (C₂₁H₁₈FN₃O₂) calcd. 363.138 found 364.4.

### Example 10

### 1-[3-(4-fluoro)-1H-pyrazolo[3,4-b]pyridin-6-yl]-3,5-dimethoxy]benzene

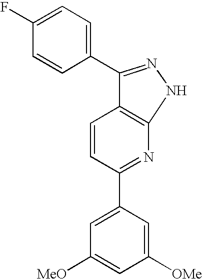

¹H NMR (600 MHz, DMSO-d₆) δ 13.89 (s, 1H), 8.60 (d, 1H, J=8.4 Hz), 8.10 (dd, 2H, J=8.4, 5.4 Hz), 7.89 (d, 1H, J=9.0 Hz), 7.36 (m, 4H), 6.64 (t, 1H, J=1.8 Hz), 3.86 (s, 6H); ¹³C NMR δ 162.8, 161.2, 160.8, 155.1, 153.0, 141.7, 140.6, 131.0, 129.7, 128.4, 116.0, 115.8, 114.9, 111.1, 55.4; MS (C₂₀H₁₆FN₃O₂) calcd. 349.122 found 350.4.

### Example 11

### 1-[3-(4-chloro-3-fluoro)-1H-pyrazolo[3,4-b]pyridin-6-yl]-3,5-dimethoxy]benzene

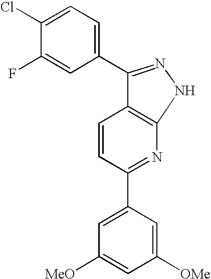

¹H NMR (600 MHz, DMSO-d₆) δ 13.89 (s, 1H), 8.66 (d, 1H, J=8.4 Hz), 8.01 (dd, 1H, J=10.2, 1.2 Hz), 7.94 (dd, 1H, J=8.4, 1.8 Hz), 7.90 (d, 2H, J=8.4 Hz), 7.34 (d, 2H, J=2.4 Hz), 6.64 (t, 1H, J=2.4 Hz), 3.86 (s, 6H); ¹³C NMR δ 160.8, 158.4, 156.8, 155.3, 153.0, 140.4, 134.2, 131.1, 123.4, 119.0, 115.2, 114.2, 111.0, 105.1, 101.6, 55.4; MS (C₂₀H₁₅ClFN₃O₂) calcd. 383.083 found 384.3.

### Example 12

### 1-[3-(4-methoxy)-1H-pyrazolo[3,4-b]pyridin-6-yl]-3,5-dimethoxy]benzene

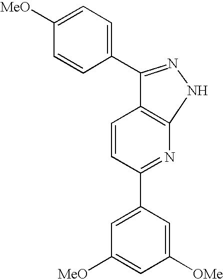

¹H NMR (600 MHz, DMSO-d₆) δ 13.73 (bs, 1H), 8.57 (dd, 1H, J=8.4, 1.8 Hz), 8.06 (d, 1H, J=7.8 Hz), 7.99 (d, 1H, J=8.4 Hz), 7.86 (d, 1H, J=8.4 Hz), 7.33 (d, 1H, J=2.4 Hz), 7.30 (d, 1H, J=8.4 Hz), 7.10 (t, 1H, J=9.0 Hz), 6.63 (t, 1H, J=1.8 Hz), 3.85 (s, 6H), 3.83 (s, 3H); ¹³C NMR δ 161.3, 160.0, 159.8, 155.4, 149.7, 141.2, 134.1, 131.6, 128.4, 128.2, 125.5, 117.6, 115.2, 114.9, 111.6, 111.3, 105.6, 102.0, 55.9, 55.7; MS (C₂₁H₁₉N₃O₃) calcd. 361.143 found 362.4.

### Example 13

### 4-[3-(4-ethoxycarbonyl)-1H-pyrazolo[3,4-b]pyridin-6-yl]-N,N-dimethyethyldiaminocarbonyl]benzene

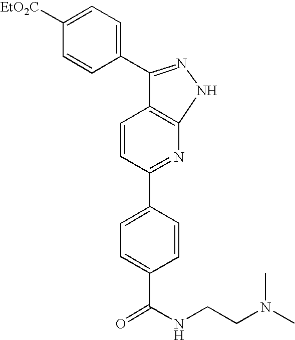

¹H NMR (600 MHz, DMSO-d₆) δ 14.14 (bs, 1H), 8.76 (d, 1H, J=9.0 Hz), 8.53 (t, 1H, J=5.4 Hz), 8.31 (d, 2H, J=8.4 Hz), 8.26 (d, 2H, J=8.4 Hz), 8.13 (d, 2H, J=8.4 Hz), 8.02 (dd, 2H, J=8.4, 1.2 Hz), 4.37 (q, 2H, J=6.6 Hz), 3.41 (m, 2H), 2.46 (m, 2H), 2.22 (s, 6H), 1.36 (t, 3H, J=7.2 Hz); ¹³C NMR δ 166.1, 166.0, 155.2, 153.7, 142.0, 141.2, 138.0, 135.7, 131.9, 130.3, 129.6, 128.2, 127.5, 126.9, 115.8, 112.0, 61.3, 58.7, 45.7, 40.5, 37.9, 14.7; MS (C₂₆H₂₇N₅O₃) calcd. 457.211 found 458.4.

### Example 14

### 3-[3-(4-ethoxycarbonyl)-1H-pyrazolo[3,4-b]pyridin-6-yl]-]benzamide

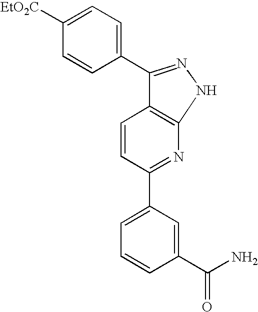

¹H NMR (600 MHz, DMSO-d₆) δ 14.13 (s, 1H), 14.04 (s, 1H), 9.90 (s, 1H), 8.77 (dd, 1H, J=8.4, 4.2 Hz), 8.71 (s, 2H), 8.37 (d, 1H, J=7.8 Hz), 8.26 (d, 1H, J=8.4), 9.19 (s, 1H), 8.16 (d, 1H, J=8.4 Hz), 8.13 (d, 1H, J=8.4 Hz), 8.00 (m, 3H), 7.64 (dt, 1H, J=6.6, 1.2 Hz), 7.49 (s, 1H), 4.37 (q, 2H, J=7.2 Hz), 1.37 (t, 3H, J=6.6 Hz); ¹³C NMR δ 168.3, 166.0, 155.5, 153.7, 142.2, 139.0, 138.0, 136.1, 135.4, 131.9, 130.3, 129.6, 129.4, 129.0, 128.2, 126.9, 126.8, 126.6115.5, 111.8, 61.3, 14.7; MS (C₂₂H₁₈N₄O₃) calcd. 386.138 found 387.4.

### Example 15

### 3-[3-(4-Fluorophenyl)-1H-pyrazolo[3,4-b]pyridin-6-yl]benzamide

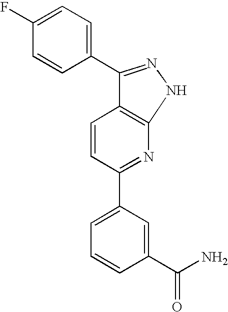

¹H NMR (600 MHz, DMSO-d₆) δ 14.13 (s, 1H), 8.72 (s, 1H), 8.66 (d, 1H, J=8.4 Hz), 8.35 (d, 1H, J=7.2 Hz), 8.20 (s, 1H), 8.11 (t, 2H, J=7.2), 8.01 (d, 1H, J=7.8 Hz), 7.96 (d, 1H, J=9.0 Hz), 7.63 (t, 1H, J=7.8 Hz), 7.50 (s, 1H), 7.38 (t, 2H, J=8.4 Hz), 7.49 (s, 1H); ¹³C NMR δ 167.7, 162.9, 161.2, 154.9, 153.1, 141.7, 138.5, 134.9, 131.3, 129.8, 129.6, 128.9, 128.5, 128.4, 126.4, 115.9, 115.8, 114.7, 111.0; MS (C₁₉H₁₃FN₄O) calcd. 332.107 found 333.3.

### Example 16

### 2-[3-(4-Chloro-3-fluorophenyl)-1H-pyrazolo[3,4-b]pyridin-6-yl]phenol

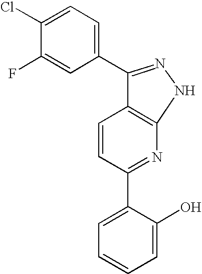

¹H NMR (600 MHz, DMSO-d₆) δ 14.01 (s, 1H), 13.10 (s, 1H), 8.72 (d, 1H, J=8.4 Hz), 8.10 (d, 1H, J=7.2 Hz), 8.04 (d, 1H, J=9.0 Hz), 7.92 (dd, 1H, J=8.4, 1.8 Hz), 7.71 (t, 1H, J=8.4 Hz), 7.36 (t, 1H, J=7.2 Hz), 6.99 (t, 2H, J=8.4 Hz); ¹³C NMR δ 159.1, 158.9, 157.2, 156.7, 150.7, 141.3, 134.3, 132.6, 132.2, 131.7, 128.9, 124.0, 120.3, 119.8, 118.3, 115.1, 114.9, 114.7, 111.2; MS (Cl₈H₁₁ClFN₃O) calcd. 339.057 found 340.4.

### Example 17

### Ethyl-4-[3-(4-methyloxyphenyl)-1H-pyrazolo[3,4-b]pyridin-6-yl]benzoate

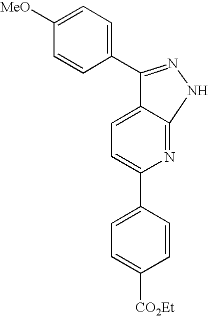

¹H NMR (600 MHz, DMSO-d₆) δ 13.79 (s, 1H), 8.66 (d, 1H, J=9.0 Hz), 8.35 (d, 1H, J=7.8 Hz), 8.13 (d, 2H, J=8.4 Hz), 8.01 (d, 2H, J=8.4 Hz), 7.94 (d, 1H, J=7.8 Hz), 7.11 (d, 2H, J=8.4 Hz), 4.37 (q, 2H, J=7.2 Hz), 3.85 (s, 3H), 1.37 (t, 3H, J=7.2 Hz).

### Example 18

### 4-{3-(4-Methyloxyphenyl)-1H-pyrazolo[3,4-b]pyridin-6-yl}acetanilide

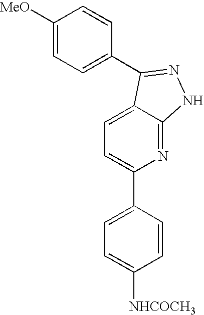

¹H NMR (600 MHz, DMSO-d₆) δ 13.64 (s, 1H), 10.14 (s, 1H), 8.56 (d, 1H, J=8.4 Hz), 8.15 (d, 2H, J=9.0 Hz), 7.99 (d, 2H, J=9.0 Hz), 7.81 (d, 1H, J=9.0 Hz), 7.76 (d, 2H, J=8.4 Hz), 7.11 (d, 2H, J=8.4 Hz), 3.85 (s, 3H), 2.10 (s, 3H).

### Example 19

### 4-[3-(4-Ethyloxycarbonylphenyl)-1H-pyrazolo[3,4-b]pyridin-6-yl]acetanilide

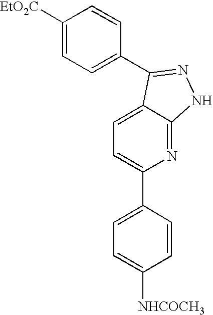

¹H NMR (600 MHz, DMSO-d₆) δ 14.00 (s, 1H), 10.16 (s, 1H), 8.67 (d, 1H, J=8.4 Hz), 8.23 (d, 2H, J=8.4 Hz), 8.11 (d, 2H, J=8.4 Hz), 8.11 (d, 2H, J=8.4 Hz), 7.89 (d, 1H, J=9.0 Hz), 7.77 (d, 2H, J=8.4 Hz), 4.37 (q, 2H, J=7.2 Hz), 2.10 (s, 3H), 1.37 (t, 3H, J=7.2 Hz).

### Example 20

### 4-[3-(4-Bromo-3-fluorophenyl)-1H-pyrazolo[3,4-b]pyridin-6-yl]acetanilide

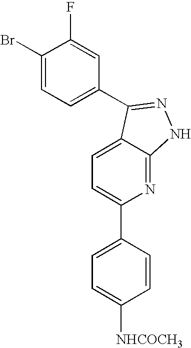

¹H NMR (600 MHz, DMSO-d₆) δ 14.00 (s, 1H), 10.10 (s, 1H), 8.72 (d, 1H, J=9.0 Hz), 8.16 (d, 1H, J=9.0 Hz), 7.96 (d, 1H, J=8.4 Hz), 7.89 (d, 1H, J=8.4 Hz), 7.72 (d, 2H, J=8.4 Hz), 7.58 (d, 2H, J=8.4 Hz), 7.38 (d, 2H, J=8.4 Hz), 2.09 (s, 3H).

### Example 21

### 4-[3-(4-Bromo-2-fluorophenyl)-1H-pyrazolo[3,4-b]pyridin-6-yl]acetanilide

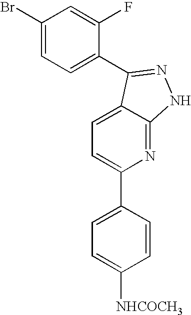

¹H NMR (600 MHz, DMSO-d₆) δ 13.13 (s, 1H), 10.10 (s, 1H), 8.35 (dd, 1H, J=8.4, 2.4 Hz), 8.08 (s, 1H), 7.94 (t, 1H, J=7.8 Hz), 7.87 (t, 1H, J=7.8 Hz), 7.82 (dd, 1H, J=9.0, 2.4 Hz), 7.77 (m, 3H), 7.35 (d, 2H, J=8.4 Hz), 2.09 (s, 3H).

### Example 22

### 4-[3-(4-Fluorophenyl)-1H-pyrazolo[3,4-b]pyridin-6-yl]acetanilide

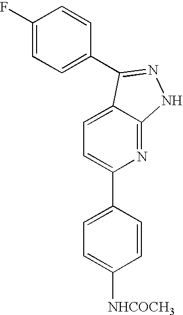

¹H NMR (600 MHz, DMSO-d₆) δ 13.78 (s, 1H), 10.15 (s, 1H), 8.59 (d, 1H, J=9.0 Hz), 8.16 (d, 2H, J=8.4 Hz), 8.10 (m, 2H), 7.84 (d, 1H, J=9.0 Hz), 7.76 (d, 2H, J=8.4 Hz), 7.37 (t, 2H, J=7.2), 2.10 (s, 3H).

### Example 23

### 4-[3-(4-Chloro-3-fluorophenyl)-1H-pyrazolo[3,4-b]pyridin-6-yl]acetanilide

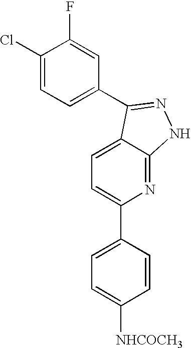

¹H NMR (600 MHz, DMSO-d₆) δ 13.95 (s, 1H), 10.15 (s, 1H), 8.67 (d, 1H, J=8.4 Hz), 8.16 (d, 2H, J=8.4 Hz), 8.02 (d, 1H, J=10.8 Hz), 7.95 (d, 1H, J=8.4 Hz), 7.86 (d, 2H, J=8.4 Hz), 7.75 (m, 3H), 2.10 (s, 3H).

### Example 24

### 4-[3-(4-Cyanophenyl)-1H-pyrazolo[3,4-b]pyridin-6-yl]acetanilide

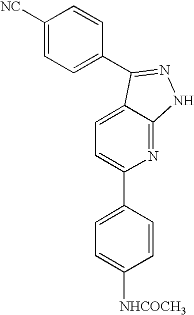

¹H NMR (600 MHz, DMSO-d₆) δ 10.16 (s, 1H), 8.69 (d, 1H, J=9.0 Hz), 8.27 (d, 2H, J=8.4 Hz), 8.17 (d, 2H, J=8.4 Hz), 7.99 (d, 2H, J=8.4 Hz), 7.89 (d, 1H, J=9.0 Hz), 7.77 (d, 2H, J=8.4 Hz), 2.10 (s, 3H).

### Example 25

### 4-[3-(3,4-Dichlorophenyl)-1H-pyrazolo[3,4-b]pyridin-6-yl]acetanilide

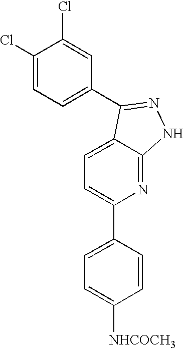

¹H NMR (600 MHz, DMSO-d₆) δ 13.98 (s, 1H), 10.16 (s, 1H), 8.64 (d, 1H, J=8.4 Hz), 8.22 (s, 1H), 8.16 (d, 2H, J=8.4 Hz), 8.06 (dd, 1H, J=7.8, 1.2 Hz), 7.87 (d, 1H, J=8.4 Hz), 7.77 (m, 3H), 2.10 (s, 3H).

### Example 26

### 4-[3-(2,4-Dichlorophenyl)-1H-pyrazolo[3,4-b]pyridin-6-yl]acetanilide

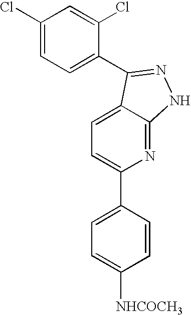

¹H NMR (600 MHz, DMSO-d₆) δ 13.99 (s, 1H), 10.15 (s, 1H), 8.18 (d, 1H, J=8.4 Hz), 8.15 (d, 2H, J=8.4 Hz), 7.85 (s, 1H), 7.82 (d, 1H, J=9.0 Hz), 7.75 (d, 2H, J=8.4 Hz), 7.71 (d, 1H, J=8.4 Hz), 7.60 (dd, 1H, J=8.4, 1.2 Hz), 2.10 (s, 3H).

### Example 27

### 4-[3-(4-Bromophenyl)-1H-pyrazolo[3,4-b]pyridin-6-yl]acetanilide

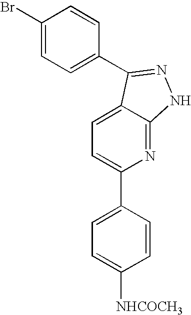

¹H NMR (600 MHz, DMSO-d₆) δ 14.03 (s, 1H), 10.07 (s, 1H), 8.67 (d, 1H, J=8.4 Hz), 8.09 (d, 2H, J=8.4 Hz), 7.81 (d, 1H, J=8.4 Hz), 7.72 (m, 5H), 7.36 (d, 1H, J=8.4 Hz), 2.08 (s, 3H).

### Example 28

### 4-[3-(3-Fluoro-4-methylphenyl)-1H-pyrazolo[3,4-b]pyridin-6-yl]acetanilide

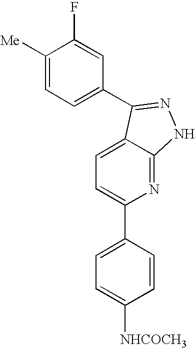

¹H NMR (600 MHz, DMSO-d₆) δ 13.83 (s, 1H), 10.16 (s, 1H), 8.62 (d, 1H, J=8.4 Hz), 8.15 (d, 2H, J=8.4 Hz), 7.77 (m, 5H), 7.45 (d, 2H, J=8.4 Hz), 2.33 (s, 3H), 2.10 (s, 3H).

### Example 29

### 4-[3-(4-Methylphenyl)-1H-pyrazolo[3,4-b]pyridin-6-yl]acetanilide

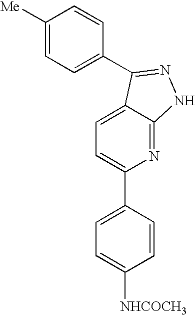

¹H NMR (600 MHz, DMSO-d₆) δ 13.71 (s, 1H), 10.16 (s, 1H), 8.58 (d, 1H, J=9.0 Hz), 8.15 (d, 2H, J=9.0 Hz), 7.94 (d, 2H, J=8.4 Hz), 7.82 (d, 1H, J=8.4 Hz), 7.74 (d, 1H, J=8.4 Hz), 7.36 (d, 1H, J=7.8 Hz), 2.93 (s, 3H), 2.10 (s, 3H).

### Example 30

### 4-[3-(2,4-Dimethylphenyl)-1H-pyrazolo[3,4-b]pyridin-6-yl]acetanilide

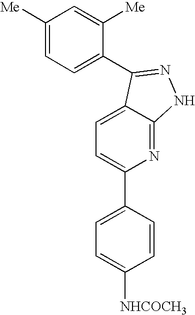

¹H NMR (600 MHz, DMSO-d₆) δ 13.69 (s, 1H), 10.14 (s, 1H), 8.14 (t, 3H, J=9.0 Hz), 7.76 (t, 3H, J=8.4 Hz), 7.45 (d, 1H, J=7.8 Hz), 7.22 (s, 1H), 7.17 (d, 1H, J=7.8 Hz), 2.39 (s, 3H), 2.37 (s, 3H), 2.10 (s, 3H).

### Example 31

### 4-[{3-(3-Methyloxyphenyl)-1H-pyrazolo[3,4-b]pyridin-6-yl}-3-methyloxy]phenol

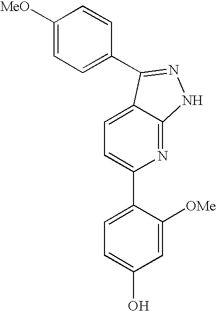

¹H NMR (600 MHz, DMSO-d₆) δ 13.96 (s, 1H), 9.48 (s, 1H), 8.62 (d, 1H, J=9.0 Hz), 8.22 (d, 2H, J=8.4 Hz), 8.11 (d, 2H, J=8.4 Hz), 7.87 (d, 1H, J=8.4 Hz), 7.80 (s, 1H), 7.67 (dd, 1H, J=7.8, 1.2 Hz), 6.94 (d, 1H, J=8.4 Hz), 2.39 (s, 3H), 4.37 (q, 2H, J=7.2 Hz), 3.91 (s, 3H), 1.37 (t, 3H, J=7.2 Hz).

### Example 32

### Ethyl-4-[3-(4-methoxycarbonyl)-1H-pyrazolo[3,4-b]pyridin-6-yl]-]benzoate

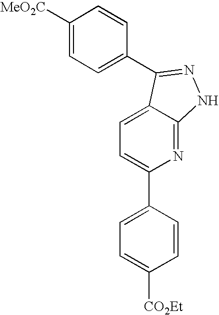

¹H NMR (600 MHz, DMSO-d₆) δ 12.80 (s, 1H), 8.56 (d, 1H, J=8.4 Hz), 8.33 (d, 1H, J=8.4 Hz), 8.10 (d, 1H, J=8.4 Hz), 8.00 (d, 1H, J=8.4 Hz), 7.94 (d, 2H, J=8.4 Hz), 7.30 (d, 1H, J=8.4 Hz), 7.09 (d, 2H, J=8.4 Hz), 7.04 (d, 1H, J=8.4 Hz), 2.39 (s, 3H), 4.36 (q, 2H, J=7.2 Hz), 3.83 (s, 3H), 1.37 (t, 3H, J=6.6 Hz).

### Example 33

### Diphenyl-4-{3-(4-ethoxycarbonylphenyl)-1H-pyrazolo[3,4-b]pyridin-6-yl}ketone

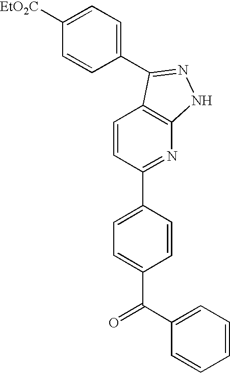

¹H NMR (600 MHz, DMSO-d₆) δ 14.00 (s, 1H), 8.64 (d, 1H, J=8.4 Hz), 8.36 (d, 2H, J=8.4 Hz), 7.97 (d, 2H, J=9.0 Hz), 7.93 (d, 1H, J=8.4 Hz), 7.89 (d, 2H, J=8.4 Hz), 7.78 (d, 2H, J=7.8 Hz), 7.69 (t, 1H, J=7.8 Hz), 7.58 (t, 2H, J=7.8 Hz), 7.08 (d, 2H, J=9.0 Hz), 4.37 ((q, 2H, J=7.2 Hz), 1.37 (t, 3H, J=7.2 Hz).

### Example 34

Diphenyl-4-{3-(4-methyloxyphenyl)-1H-pyrazolo[3,4-b]pyridin-6-yl}ketone

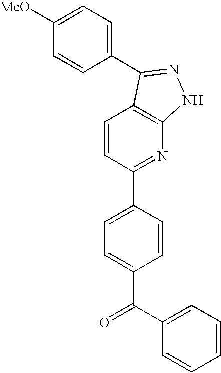

¹H NMR (600 MHz, DMSO-d₆) δ 13.89 (s, 1H), 8.64 (d, 1H, J=8.4 Hz), 8.36 (d, 2H, J=8.4 Hz), 7.97 (d, 2H, J=9.0 Hz), 7.93 (d, 1H, J=8.4 Hz), 7.89 (d, 2H, J=8.4 Hz), 7.78 (d, 2H, J=7.8 Hz), 7.69 (t, 1H, J=7.8 Hz), 7.58 (t, 2H, J=7.8 Hz), 7.08 (d, 2H, J=9.0 Hz), 3.81 (s, 3H).

### Example 35

### Diphenyl-4-{3-(4-chloro-3-fluorophenyl)-1H-pyrazolo[3,4-b]pyridin-6-yl}ketone

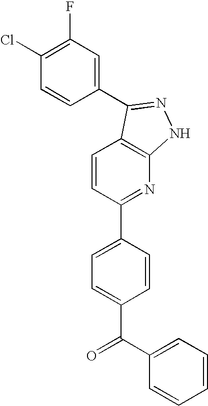

¹H NMR (600 MHz, DMSO-d₆) δ 14.01 (s, 1H), 8.79 (d, 1H, J=8.4 Hz), 8.70 (d, 2H, J=8.4 Hz), 8.40 (d, 2H, J=8.4 Hz), 8.12 (m, 2H), 7.91 (d, 2H, J=8.4 Hz), 7.80 (m, 2H), 7.36 (d, 2H, J=8.4 Hz), 7.32 (t, 1H, J=7.8 Hz).

### Example 36

### Diphenyl-4-{3-(4-fluorophenyl)-1H-pyrazolo[3,4-b]pyridin-6-yl}ketone

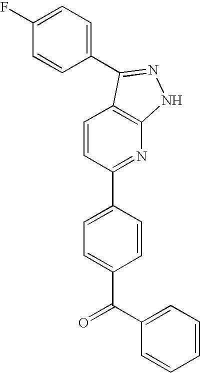

¹H NMR (600 MHz, DMSO-d₆) δ 13.70 (s, 1H), 8.71 (d, 1H, J=8.4 Hz), 8.39 (d, 2H, J=8.4 Hz), 8.12 (m, 1H), 8.00 (d, 1H, J=9.0 Hz), 7.95 (m, 1H), 7.91 (d, 2H, J=8.4 Hz), 7.81 (d, 2H, J=7.8 Hz), 7.72 (t, 1H, J=7.2 Hz), 7.60 (t, 2H, J=7.2 Hz), 7.38 (t, 2H, J=9.0 Hz), 7.29 (t, 1H, J=9.0 Hz).

### Example 37

### Diphenyl-4-{3-(3-fluoro-4-methylphenyl)-1H-pyrazolo[3,4-b]pyridin-6-yl}ketone

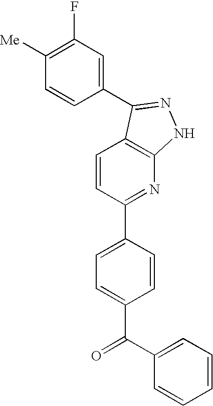

¹H NMR (600 MHz, DMSO-d₆) δ 13.99 (s, 1H), 8.72 (d, 1H, J=8.4 Hz), 8.39 (d, 2H, J=8.4 Hz), 7.99 (d, 1H, J=8.4 Hz), 7.92 (d, 2H, J=8.4 Hz), 7.80 (m, 4H), 7.60 (t, 2H, J=7.8 Hz), 7.46 (t, 1H, J=7.8 Hz), 2.32 (s, 3H).

### Example 38

### Diphenyl-4-{3-(4-bromo-3-fluorophenyl)-1H-pyrazolo[3,4-b]pyridin-6-yl}ketone

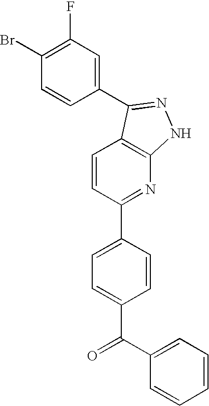

¹H NMR (600 MHz, DMSO-d₆) δ 14.01 (s, 1H), 8.69 (d, 1H, J=8.4 Hz), 8.70 (d, 2H, J=8.4 Hz), 8.40 (d, 2H, J=8.4 Hz), 8.12 (m, 2H), 7.91 (d, 2H, J=8.4 Hz), 7.80 (m, 2H), 7.36 (d, 2H, J=8.4 Hz), 7.32 (t, 1H, J=7.8 Hz).

### Example 39

Diphenyl-4-{3-(4-bromo-2-fluorophenyl)-1H-pyrazolo[3,4-b]pyridin-6-yl}ketone

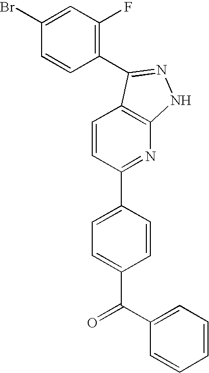

¹H NMR (600 MHz, DMSO-d₆) δ 13.13 (s, 1H), 8.71 (d, 1H, J=8.4 Hz), 8.39 (d, 2H, J=8.4 Hz), 8.35 (d, 1H, J=8.4 Hz), 8.12 (m, 2H), 8.08 (s, 1H), 7.91 (d, 2H, J=8.4 Hz), 7.80 (m, 2H), 7.36 (d, 2H, J=8.4 Hz), 7.32 (t, 1H, J=7.8 Hz).

### Example 40

### 3-[3-(2,4-Dichlorophenyl)-1H-pyrazolo[3,4-b]pyridin-6-yl]acetanilide

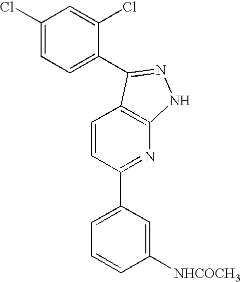

¹H NMR (600 MHz, DMSO-d₆) δ 13.99 (s, 1H), 10.12 (s, 1H), 8.45 (s, 1H), 8.22 (d, 1H, J=8.4 Hz), 7.85 (d, 1H, J=1.8 Hz), 7.81 (d, 1H, J=7.8 Hz), 7.78 (d, 1H, J=8.4 Hz), 7.70 (m, 2H), 7.60 (dd, 1H, J=8.4, 1.8 Hz), 7.45 (t, 1H, J=7.8 Hz), 2.06 (s, 3H).

### Example 41

### 3-[3-(4-Bromo-2-fluorophenyl)-1H-pyrazolo[3,4-b]pyridin-6-yl]acetanilide

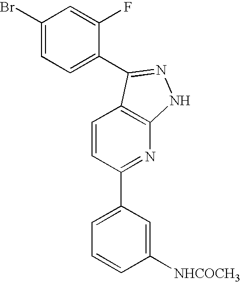

¹H NMR (600 MHz, DMSO-d₆) δ 13.45 (s, 1H), 10.03 (s, 1H), 8.45 (s, 1H), 8.38 (dd, 1H, J=8.4, 2.4 Hz), 8.15 (d, 1H, J=7.8 Hz), 8.08 (s, 1H), 7.78 (d, 1H, J=8.4 Hz), 7.70 (m, 2H), 7.60 (dd, 1H, J=8.4, 1.8 Hz), 7.45 (t, 1H, J=7.8 Hz), 2.06 (s, 3H).

### Example 42

### 2-[3-(3,4-Dichlorophenyl)-1H-pyrazolo[3,4-b]pyridin-6-yl]acetanilide

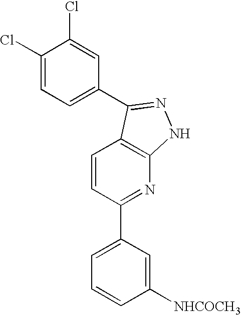

¹H NMR (600 MHz, DMSO-d₆) δ 13.98 (s, 1H), 10.13 (s, 1H), 8.69 (d, 1H, J=8.4 Hz), 8.45 (s, 1H), 8.23 (d, 1H, J=1.8 Hz), 8.06 (dd, 1H, J=7.8, 1.8 Hz), 7.81 (m, 3H), 7.71 (d, 1H, J=7.8 Hz), 7.46 (t, 1H, J=7.8 Hz), 2.09 (s, 3H).

### Example 43

### 3-[3-(4-Bromo-3-fluorophenyl)-1H-pyrazolo[3,4-b]pyridin-6-yl]acetanilide

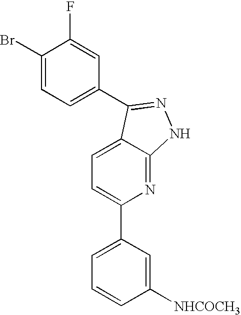

¹H NMR (600 MHz, DMSO-d₆) δ 14.00 (s, 1H), 10.07 (s, 1H), 8.72 (d, 1H, J=9.0 Hz), 8.15 (d, 1H, J=9.0 Hz), 7.96 (d, 1H, J=8.4 Hz), 7.92 (d, 1H, J=8.4 Hz), 7.88 (s, 1H), 7.78 (m, 1H), 7.42 (d, 1H, J=8.4 Hz), 7.37 (d, 1H, J=8.4 Hz), 7.28 (d, 1H, J=8.4 Hz), 2.07 (s, 3H).

### Example 44

### 3-[3-(4-Cyanophenyl)-1H-pyrazolo[3,4-b]pyridin-6-yl]acetanilide

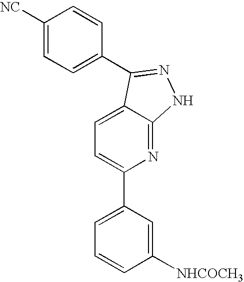

¹H NMR (600 MHz, DMSO-d₆) δ 10.13 (s, 1H), 8.74 (d, 1H, J=8.4 Hz), 8.46 (s, 1H), 8.28 (d, 2H, J=8.4 Hz), 7.99 (d, 2H, J=8.4 Hz), 7.85 (d, 2H, J=8.4 Hz), 7.71 (d, 1H, J=7.8 Hz), 7.46 (t, 1H, J=7.8 Hz), 2.09 (s, 3H).

### Example 45

### 3-[3-(4-Bromophenyl)-1H-pyrazolo[3,4-b]pyridin-6-yl]acetanilide

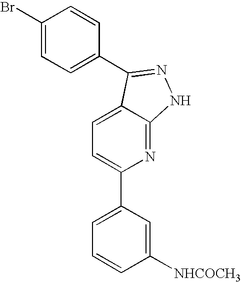

¹H NMR (600 MHz, DMSO-d₆) δ 10.06 (s, 1H), 8.68 (d, 1H, J=8.4 Hz), 8.31 (s, 1H), 8.12 (d, 2H, J=8.4 Hz), 7.99 (s, 1H), 7.77 (d, 2H, J=8.4 Hz), 7.58 (d, 1H, J=7.8 Hz), 7.45 (t, 1H, J=7.8 Hz), 7.36 (d, 1H, J=7.8 Hz), 2.08 (s, 3H).

### Example 46

### 3-[3-(4-Ethylphenyl)-1H-pyrazolo[3,4-b]pyridin-6-yl]acetanilide

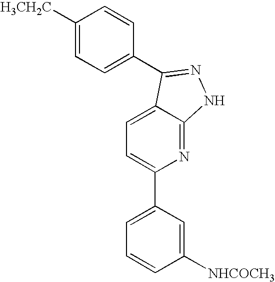

¹H NMR (600 MHz, DMSO-d₆) δ 10.12 (s, 1H), 8.63 (d, 1H, J=8.4 Hz), 8.45 (s, 1H), 7.97 (d, 2H, J=7.8 Hz), 7.83 (d, 1H, J=7.8 Hz), 7.77 (d, 1H, J=8.4 Hz), 7.70 (d, 1H, J=7.8 Hz), 7.45 (t, 1H, J=8.4 Hz), 7.39 (d, 1H, J=8.4 Hz), 2.68 (q, 2H, J=7.8 Hz), 2.09 (s, 3H), 1.24 (t, 3H, J=7.2 Hz).

### Example 47

### 3-[3-(2,4-Dimethylphenyl)-1H-pyrazolo[3,4-b]pyridin-6-yl]acetanilide

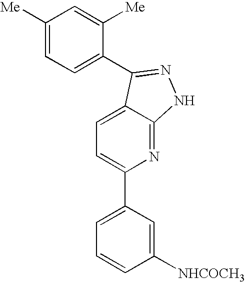

¹H NMR (600 MHz, DMSO-d₆) δ 10.12 (s, 1H), 8.44 (s, 1H), 8.20 (d, 1H, J=8.4 Hz), 7.81 (d, 1H, J=7.2 Hz), 7.22 (s, 1H), 7.72 (d, 1H, J=8.4 Hz), 7.70 (d, 1H, J=7.8 Hz), 7.46 (d, 2H, J=7.8 Hz), 7.22 (s, 1H), 2.39 (s, 3H), 2.37 (s, 3H), 2.09 (s, 3H).

### Example 48

### Ethyl-4-[3-(4-ethyloxycarbonylphenyl)-1H-pyrazolo[3,4-b]pyridin-6-yl]benzoate

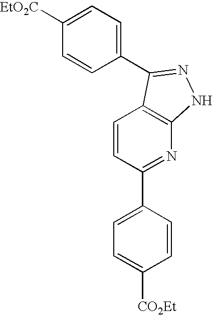

¹H NMR (600 MHz, DMSO-d₆) δ 14.17 (s, 1H), 8.76 (d, 1H, J=9.0 Hz), 8.36 (d, 2H, J=8.4 Hz), 8.24 (d, 2H, J=8.4 Hz), 8.12 (m, 4H), 7.22 (s, 1H), 8.00 (d, 1H, J=8.4 Hz), 4.36 (dq, 4H, J=7.2, 1.2 Hz), 1.37 (t, 6H, J=6.6 Hz).

### Example 49

### Ethyl-4-[3-(4-chloro-3-fluorophenyl)-1H-pyrazolo[3,4-b]pyridin-6-yl]benzoate

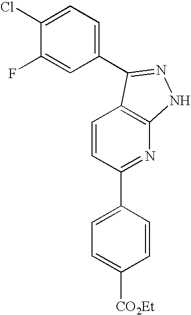

¹H NMR (600 MHz, DMSO-d₆) δ 14.19 (s, 1H), 8.78 (d, 1H, J=8.4 Hz), 8.37 (d, 2H, J=8.4 Hz), 8.13 (d, 2H, J=8.4 Hz), 8.05 (dd, 1H, J=10.8, 1.8 Hz), 8.00 (d, 1H, J=8.4 Hz), 7.97 (dd, 1H, J=8.4, 1.2 Hz), 7.75 (t, 1H, J=7.8 Hz), 4.36 (q, 2H, J=7.2 Hz), 1.37 (t, 3H, J=7.2 Hz).

### Example 50

### Ethyl-4-[3-(4-bromo-3-fluorophenyl)-1H-pyrazolo[3,4-b]pyridin-6-yl]benzoate

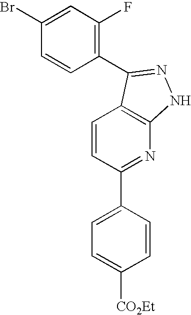

¹H NMR (600 MHz, DMSO-d₆) δ 13.19 (s, 1H), 8.11 (s, 1H), 8.08 (d, 2H, J=8.4 Hz), 7.95 (dd, 3H, J=10.8, 1.8 Hz), 7.84 (dd, 1H, J=8.4, 1.2 Hz), 7.80 (d, 2H, J=9.0 Hz), 7.73 (dd, 1H, J=8.4, 1.2 Hz), 4.36 (q, 2H, J=6.6 Hz), 1.36 (t, 3H, J=7.2 Hz).

### Example 51

### Ethyl-4-[3-(3-fluoro-4-methylphenyl)-1H-pyrazolo[3,4-b]pyridin-6-yl]benzoate

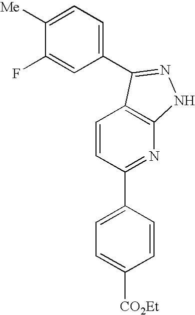

¹H NMR (600 MHz, DMSO-d₆) δ 13.89 (s, 1H), 8.72 (d, 1H, J=9.0 Hz), 8.35 (d, 2H, J=8.4 Hz), 8.12 (d, 2H, J=8.4 Hz), 7.96 (d, 1H, J=9.0 Hz), 7.83 (d, 1H, J=7.8 Hz), 7.77 (d, 1H, J=10.8 Hz), 4.36 (q, 2H, J=7.2 Hz), 2.32 (s, 3H), 1.37 (t, 3H, J=7.2 Hz).

### Example 52

### Ethyl-4-[3-(4-fluorophenyl)-1H-pyrazolo[3,4-b]pyridin-6-yl]benzoate

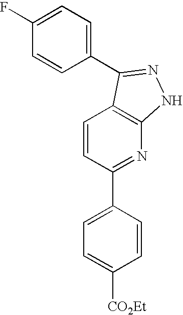

¹H NMR (600 MHz, DMSO-d₆) δ 13.80 (s, 1H), 8.69 (d, 1H, J=8.4 Hz), 8.35 (d, 2H, J=8.4 Hz), 8.11 (m, 4H), 7.96 (d, 1H, J=8.4 Hz), 7.38 (t, 1H, J=9.0 Hz), 4.36 (q, 2H, J=7.2 Hz), 1.37 (t, 3H, J=7.2 Hz).

### Example 53

### Ethyl-4-[3-(3,4-dichlorophenyl)-1H-pyrazolo[3,4-b]pyridin-6-yl]benzoate

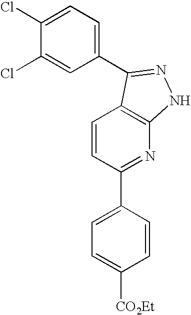

¹H NMR (600 MHz, DMSO-d₆) δ 13.89 (s, 1H), 8.75 (d, 1H, J=8.4 Hz), 8.36 (d, 2H, J=8.4 Hz), 8.24 (d, 1H, J=1.8 Hz), 8.13 (d, 2H, J=8.4 Hz), 8.08 (dd, 1H, J=8.4, 1.8 Hz), 7.99 (d, 1H, J=8.4 Hz), 7.80 (d, 1H, J=8.4 Hz), 4.37 (q, 2H, J=6.6 Hz), 1.37 (t, 3H, J=7.2 Hz).

### Example 54

### Ethyl-4-[3-(4-cyanophenyl)-1H-pyrazolo[3,4-b]pyridin-6-yl]benzoate

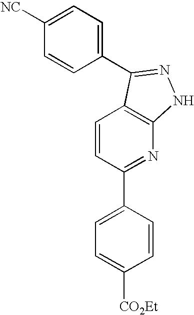

¹H NMR (600 MHz, DMSO-d₆) δ 13.15 (s, 1H), 8.71 (d, 1H, J=8.4 Hz), 8.22 (d, 2H, J=8.4 Hz), 8.14 (m, 2H), 7.99 (m, 3H), 7.91 (d, 1H, J=8.4 Hz), 7.40 (d, 1H, J=8.4 Hz), 4.36 (q, 2H, J=7.2 Hz), 1.37 (t, 3H, J=7.2 Hz).

### Example 55

### Ethyl-4-[3-(4-bromophenyl)-1H-pyrazolo[3,4-b]pyridin-6-yl]benzoate

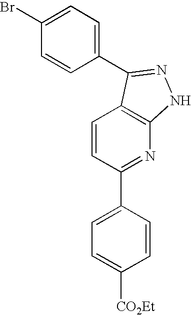

¹H NMR (600 MHz, DMSO-d₆) δ 13.89 (s, 1H), 8.34 (d, 2H, J=8.4 Hz), 8.24 (d, 1H, J=8.4 Hz), 8.12 (d, 2H, J=8.4 Hz), 7.95 (d, 1H, J=8.4 Hz), 7.86 (d, 1H, J=2.4 Hz), 7.72 (d, 1H, J=8.4 Hz), 7.61 (dd, 1H, J=8.4, 1.8 Hz), 4.37 (q, 2H, J=6.6 Hz), 1.37 (t, 3H, J=7.2 Hz).

### Example 56

### 6-[3-(4-Ethyloxycarbonylphenyl)-1H-pyrazolo[3,4-b]pyridin-6-yl]-1H-indole

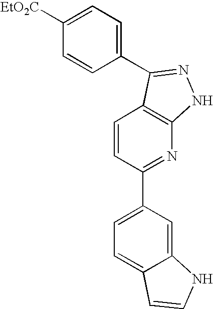

¹H NMR (600 MHz, DMSO-d₆) δ 9.09 (s, 1H), 8.70 (d, 2H, J=9.0 Hz), 8.57 (d, 1H, J=8.4 Hz), 8.22 (d, 2H, J=8.4 Hz), 8.12 (m, 2H), 7.70 (d, 1H, J=8.4 Hz), 7.61 (dd, 1H, J=8.4, 1.8 Hz), 6.98 (d, 1H, J=2.4 Hz), 6.82 (dd, 1H, J=8.4, 1.8 Hz), 6.66 (d, 1H, J=3.0 Hz), 4.36 (q, 2H, J=7.2 Hz), 1.36 (t, 3H, J=7.2 Hz).

The following compounds can generally be made using the methods described above. It is expected that these compounds when made will have activity similar to those that have been made in the examples above.

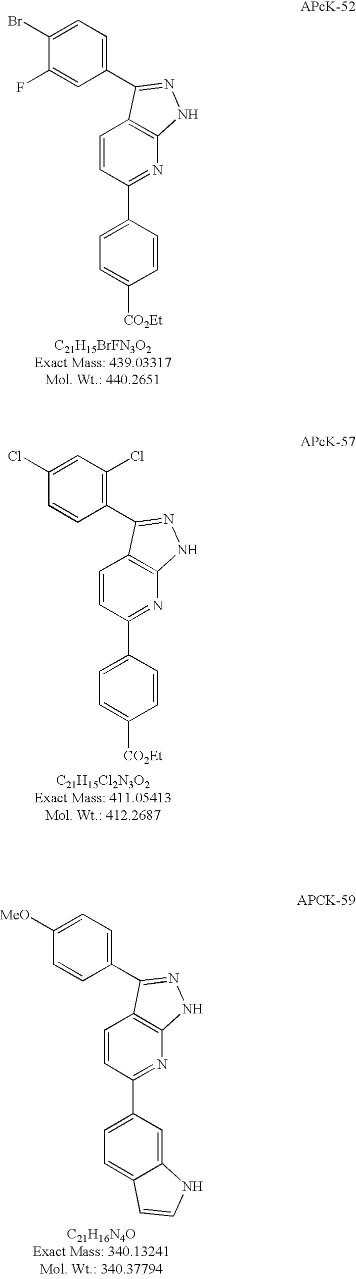

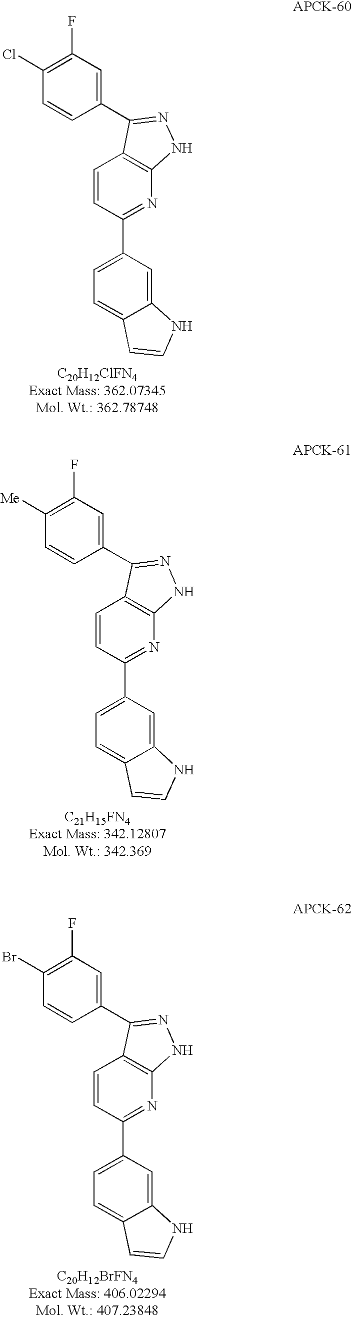

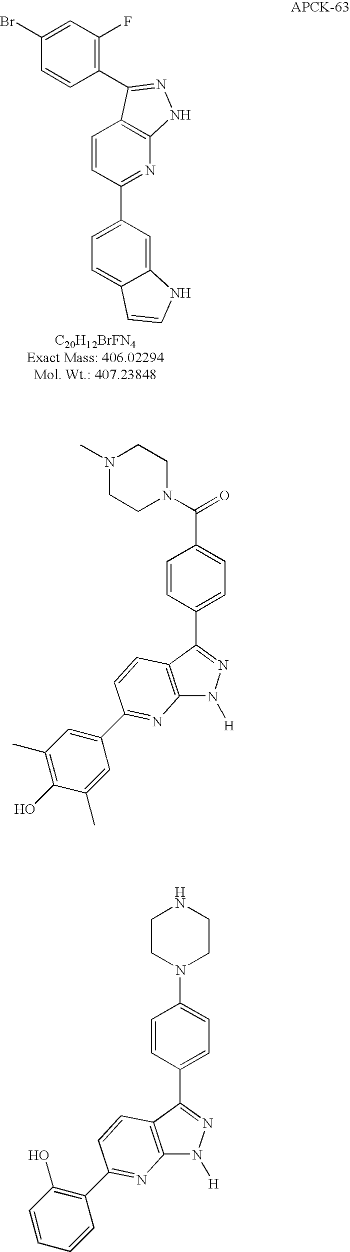

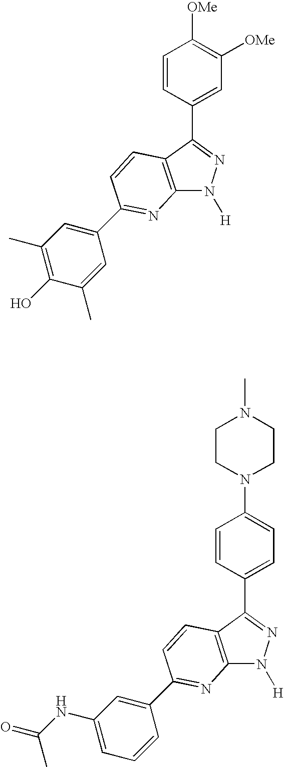

The activity of the compounds as NO Synthase inhibitors in Examples 1-56 has been shown by the following assays. The other compounds listed above, which have not yet been made, are predicted to have activity in these assays as well.

### Biological Activity Assay

### Enzymatic Assays of c-Kit Activity in Presence of Inhibitors

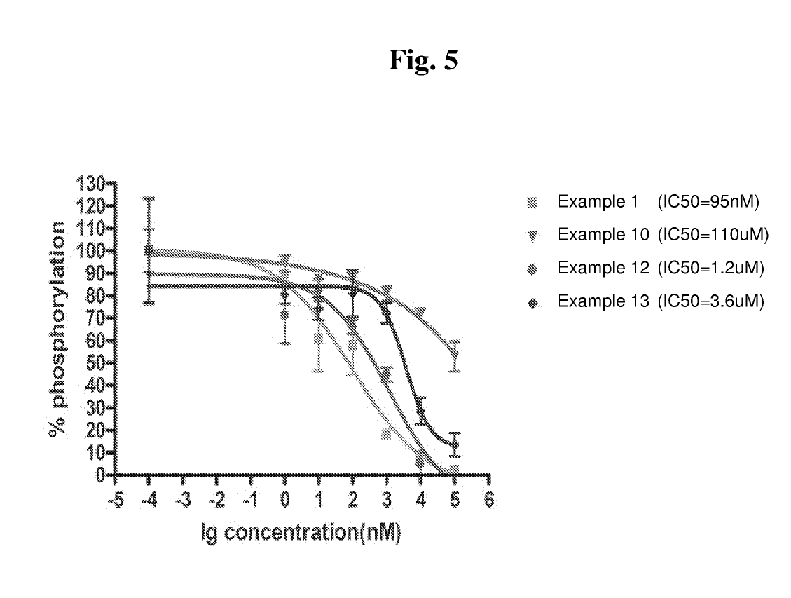

IC50 values were determined for compounds of Examples 1, 10, 12 and 13 using a c-Kit kinase enzymatic assay. Phosphorylation of tyrosine was measured using a kinase assay protocol as provided by UpState Cell Signaling Solutions (Lake Placid, N.Y.). An IC50 value represents the concentration of a drug that is required for 50% inhibition. The log concentration-response plot is shown in FIG. 5. The compound of Example 1 has an IC50 of about 95 nM. The compound of Example 10 has an IC50 of greater than 100 μM. The compound of Example 12 has an IC50 of about 1.2 μM. The compound of Example 13 has an IC50 of about 3.6 μM. The results of the kinase assays validate the modeling and assessment of the structure/activity relationship data.

### Biological Activity Assay

### Cell Line Assays of c-Kit Activity in Presence of Inhibitors

An MTT-(3,(4,5-dimethylthiazol-2-yl)-5-(3-carboxymethoxyphenyl)-2-(4-sulfophenyl)-2H-tetrazolium) based cell proliferation/toxicity assay system (Promega, Madison, Wis.) using cell lines, including the AML cell lines OCIM2 and OCI/AML3, was used to evaluate the effectiveness of the compounds as set forth herein in inhibition of c-Kit cellular activity.

AML cell lines OCIM2 and OCI/AML3 are human erythroleukemia lines and were provided by M. D. Minden, (Ontario Cancer Institute, Toronto, ON, Canada and as referenced in: Papayannopoulou et al., Blood (1988) 72:1029-1038; Wang et al., Leukemia (1989) 3: 236-249). Cells were maintained in RPMI 1640 culture medium (GIBCO, Grand Island, N.Y.) supplemented with 10% FCS (Flow Laboratories, McLean, Va.) and split twice weekly.

Briefly, cells were harvested at the logarithmic phase of their growth. They were then washed twice in RPMI 1640 containing 10% FCS and counted in a hemocytometer, and their viability was determined using 0.1% trypan blue staining. Equal numbers of viable cells (5×104 cells per well) were incubated in RPMI 1640 medium supplemented with 10% FCS alone or with the candidate drugs at increasing concentrations; the incubations were continued for 72 h in 96-well flat-bottomed plates (Linbro; Flow Laboratories, McLean, Va.) at 37° C. in a humidified 5% CO2 atmosphere. After incubation, 20 μl of CellTiter96 One Solution Reagent (Promega) was added to each well. The plates were then incubated for an additional 60 min at 37° C. in a humidified 5% CO2 atmosphere. Immediately after incubation, absorbance was read using a 96-well plate reader at a wavelength of 490 nm. Each data point was determined six times before analysis.

While OCIM2 and OCI/AML3 are both AML cell lines, c-Kit is expressed to a much higher degree in the OCI/AML3 line versus OCIM2, as determined by the responsiveness to stem cell factor (SCF), which mediates c-Kit receptor dimerization, activation, and autophosphorylation (Blume-Jensen, et al., (1991) EMBO Journal 10, 4121-8).

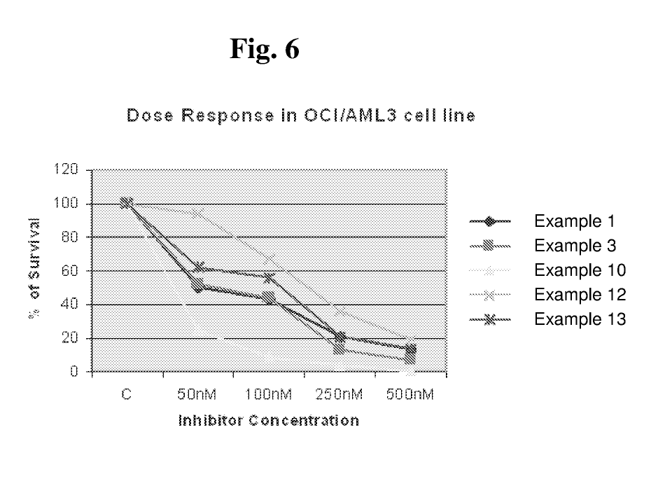

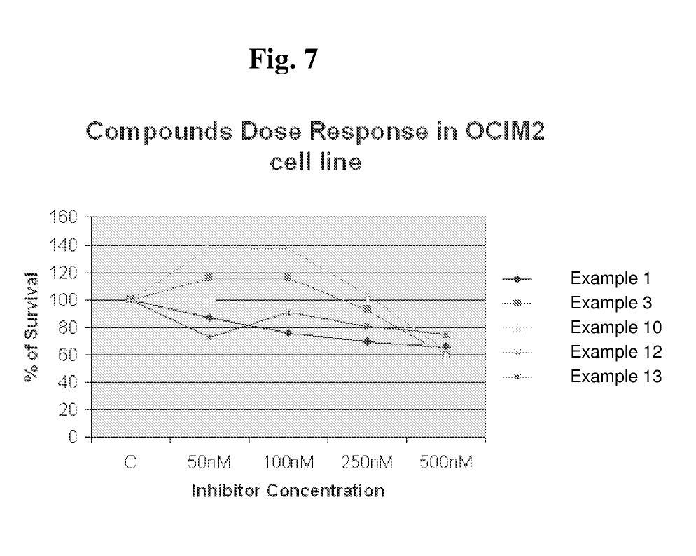

Dose-dependent results of cell viability studies in the OCI/AML3 and OCIM2 cell lines are shown in FIG. 6 and FIG. 7, respectively. 3-{(4-Methyloxyphenyl)-1H-pyrazolo[4,3-b]pyridin-6-yl}acetanilide has an EC50 of about 100 nM in the cell line OCI/AML3, and all compounds of FIG. 6 demonstrated EC50's better than 500 nM. An EC50 value represents a cellular concentration for obtaining 50% of the maximum effect. The same compounds had a significantly reduced effect on the OCIM2 cell line as shown by FIG. 7 since uninduced OCIM2 cells express much lower amounts of c-Kit. These comparative data indicate a specificity towards c-Kit.

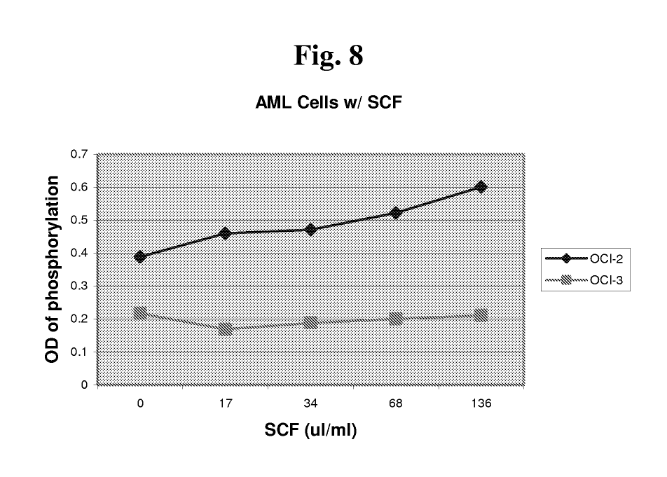

Dose-dependent results of stem cell factor induction of c-Kit kinase in the OCIM2 cell line is shown by FIG. 8. The OCI-AML3 line constitutively expresses c-Kit kinase and is essentially unresponsive to induction.

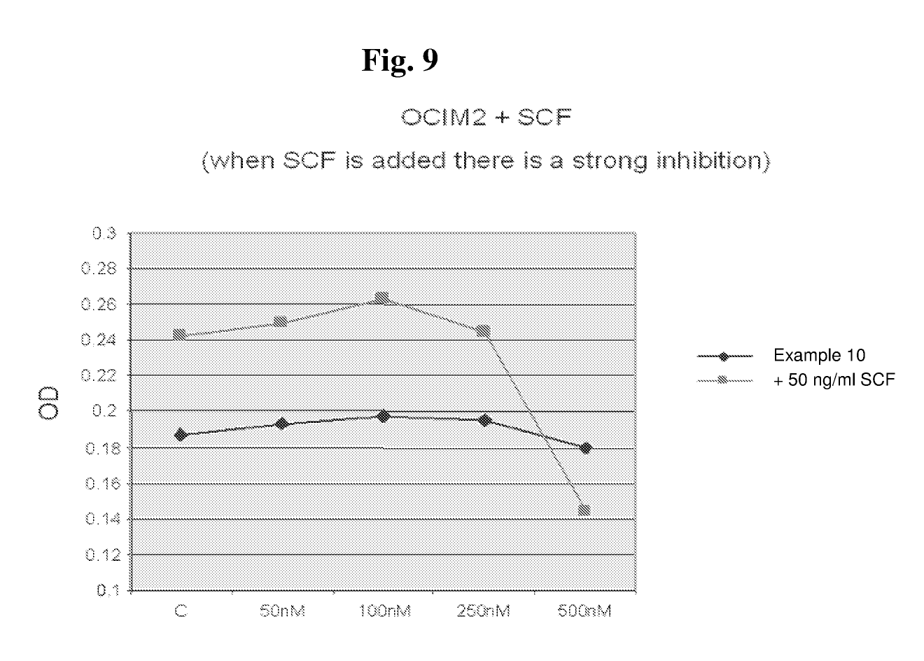

FIG. 9 shows dose-dependent inhibition of c-Kit kinase by antagonist 1-[3-(4-fluoro-3-methyl)-1H-pyrazolo[3,4-b]pyridin-6-yl]-3,5-dimethoxy]benzene in the OCIM2 cell line with and without stem cell factor induction (50 ng/mL SCF). SCF stimulates OCIM2 proliferation by binding to its cellular receptor c-Kit. When SCF is added to the culture, c-Kit is activated and stimulates OCIM2 proliferation. When 1-[3-(4-fluoro)-1H-pyrazolo[3,4-b]pyridin-6-yl]-3,5-dimethoxy]benzene is added, it is inhibiting the proliferation of these cells. The data indicate that the antagonist 1-[3-(4-chloro-3-fluoro)-1H-pyrazolo[3,4-b]pyridin-6-yl]-3,5-dimethoxy]benzene affects directly c-Kit.

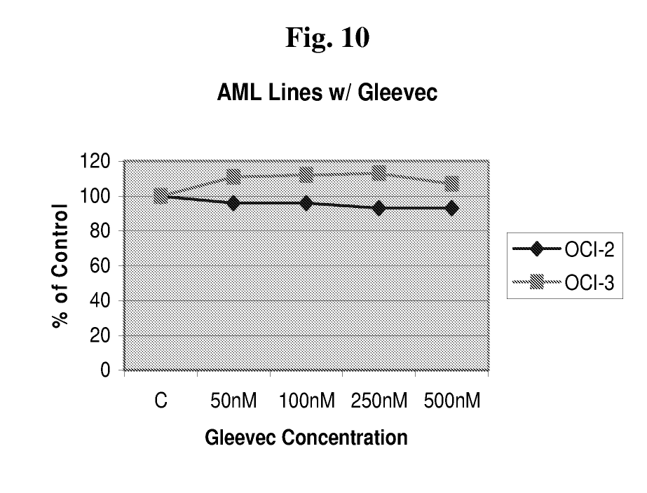

A concentration response plot for GLEEVEC® in the AML cell lines OCIM2 (uninduced) and OCI-AML3 (expressing c-Kit) is shown by FIG. 10. These cell lines are resistant to prior art compound IMATINIB® (GLEEVEC®) at concentrations at which antagonists as set forth herein possess inhibitory activity.

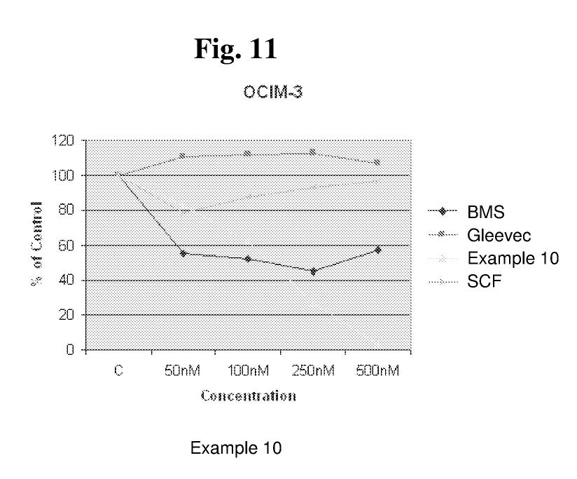

A comparison of activity of a representative c-Kit kinase antagonist of the present invention with prior art compounds in the OCI-AML3 cell line is provided by FIG. 11. The concentration response plot shows inhibitory activity of 1-[3-(4-fluoro)-1H-pyrazolo[3,4-b]pyridin-6-yl]-3,5-dimethoxy]benzene as set forth herein as compared to prior art compounds Dasatinib (BMS) and GLEEVEC® (IMATINIB®) in the OCI-AML3 line expressing c-Kit kinase.

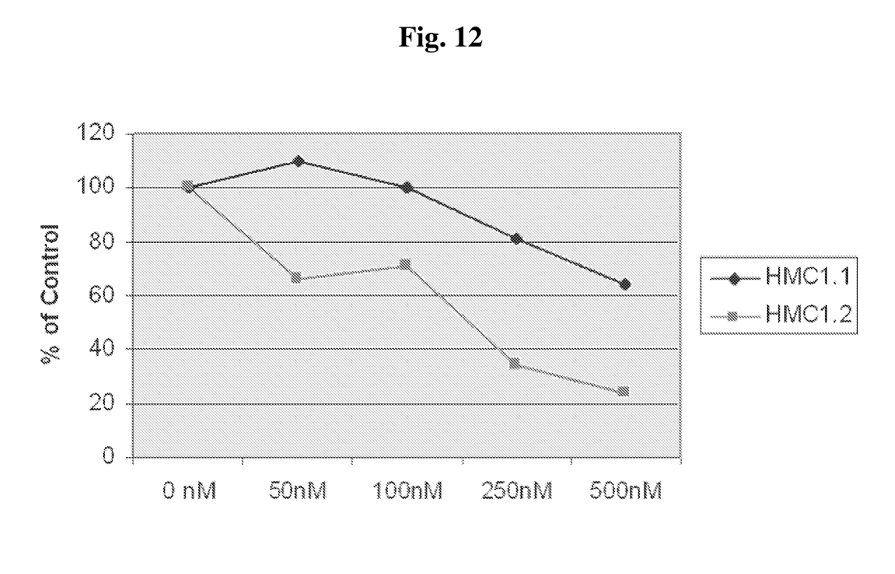

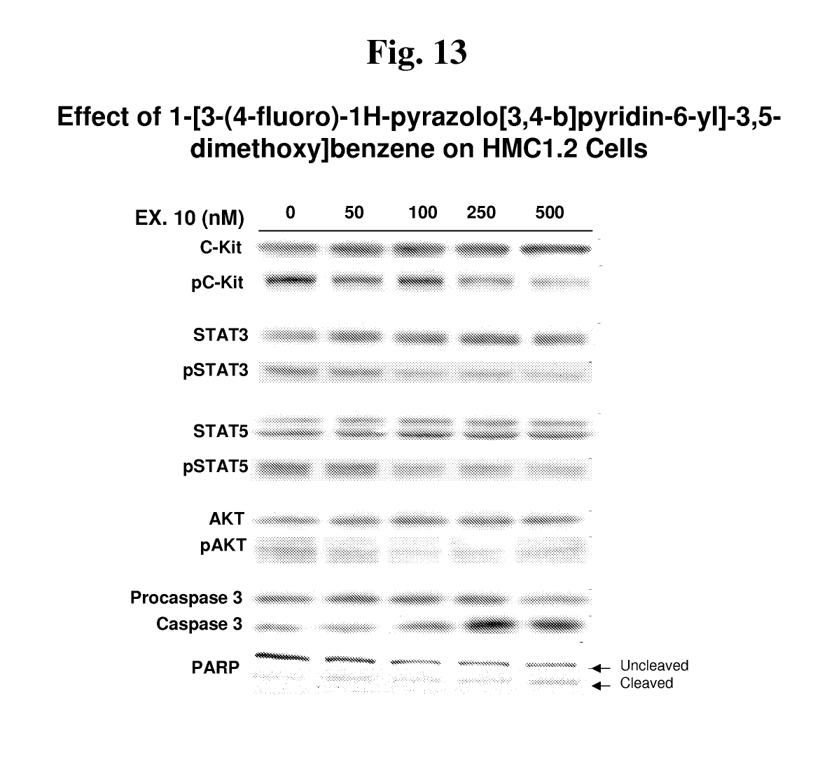

The effect of compound 1-[3-(4-fluoro)-1H-pyrazolo[3,4-b]pyridin-6-yl]-3,5-dimethoxy]benzene on two systemic mastocytosis cell lines with unmutated (wild-type) c-Kit (HCM1.1 cell line) and a cell line with mutated c-Kit (mutation at position 816) (HCM1.2 cell line) was also determined. Cell lines were obtained from Dr. Verstovsek (Leuk Res. 2006 Jun. 22, online prepublication). Assay data as shown by FIG. 12 and FIG. 13 confirm the activity of the compounds and selectivity for the cell line containing mutated c-Kit.

### Structure-Based Design of c-Kit Inhibitors

The design of c-Kit inhibitors provided herein includes the selection of screening compounds, in-silico screening via docking, analysis of docking results, preliminary selection of compounds with a degree of specificity towards c-Kit, and final selection of candidate compounds.

Compounds from three vendors: Asinex (Winston-Salem, N.C.), BioFocus (Saffron Walden, Essex, UK), and LifeChem (Burlington, ON, Canada) were screened in-silico against c-Kit and c-Met via docking the individual ligands into the receptor binding site.

The traditional metric for this type of screening has been the individual docking scores which aim to capture the binding affinity of the ligand and receptor. Though useful in ranking ligands, the scoring methods are imperfect, so the consensus of several scoring methods (Clark, et al., (2002) J Mol Graph Model 20, 281-95) is generally utilized in the final selection. In the present study, the consensus scoring was completed with the scoring methods provided in the FlexX (Kramer, et al., (1999) Proteins 37, 228-41) module within Sybyl 7.1 (Tripos, Inc., St. Louis, Mo.). Over 32,000 compounds were screened in this manner. While compounds with the highest scores were of interest, additional metrics were also considered. For example, an emphasis was placed on identifying those compounds with a larger difference in scores when compared to other kinases. The underlying principle is that selections based on the larger score differences should translate into greater selectivity towards one kinase.

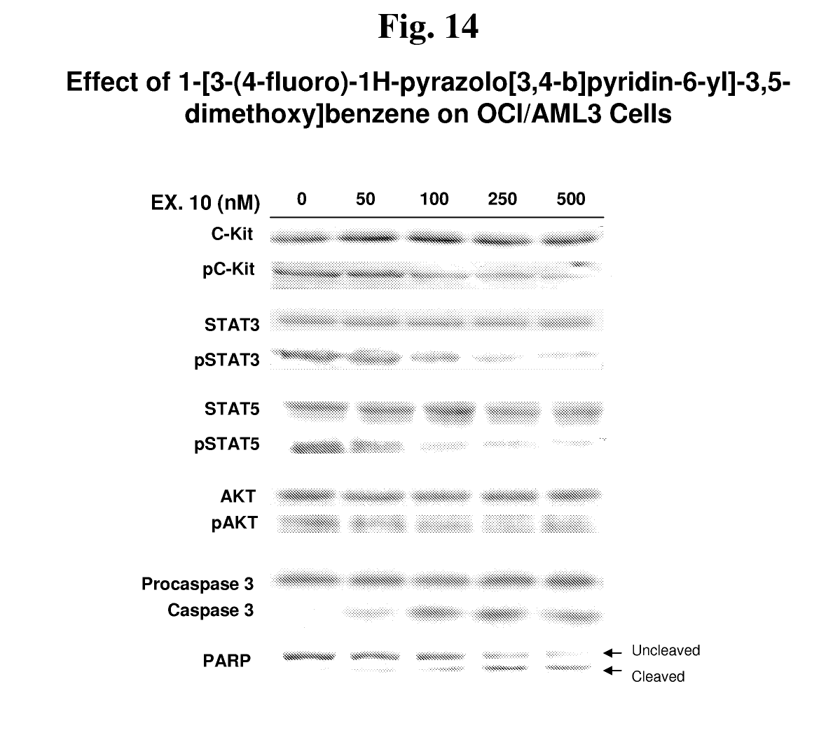

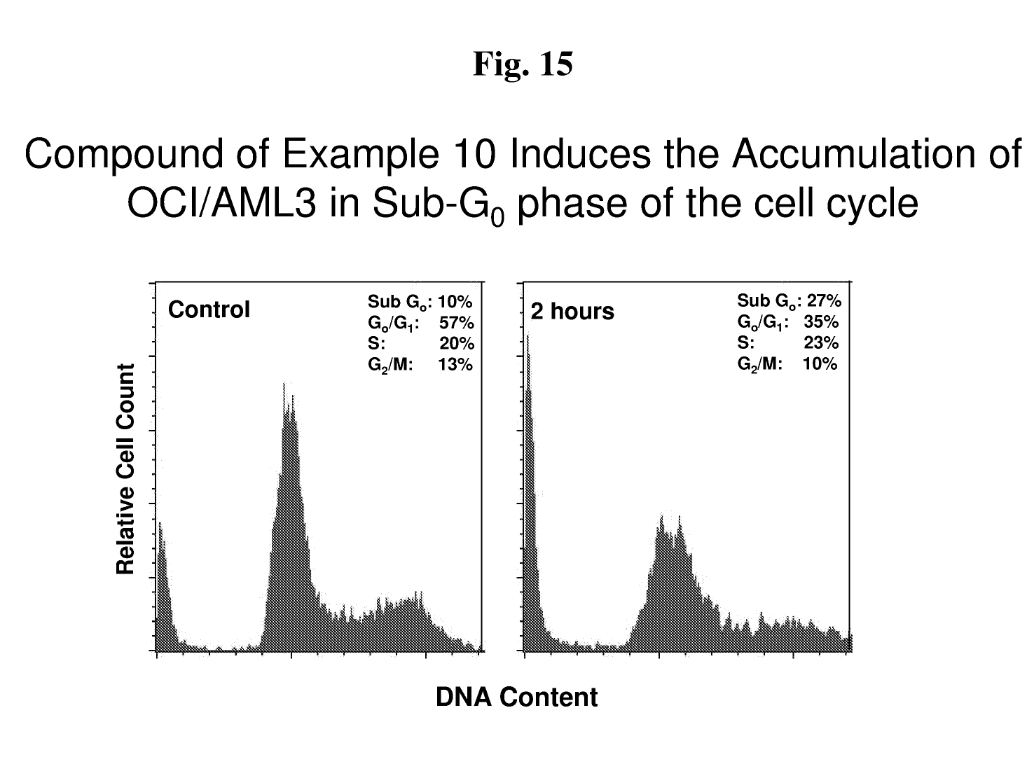

FIGS. 13 and 14 show the effect of the compound of Example 10 on a constitutively activated C-Kit cell line (FIG. 13) and in acute leukemia cell line OCI/AML3 (Ontario Cancer Institute, FIG. 14). FIG. 15 shows the compound of Example 10 causes the accumulation of OCI/AML3 in Sub-Go phase in the cell cycle.

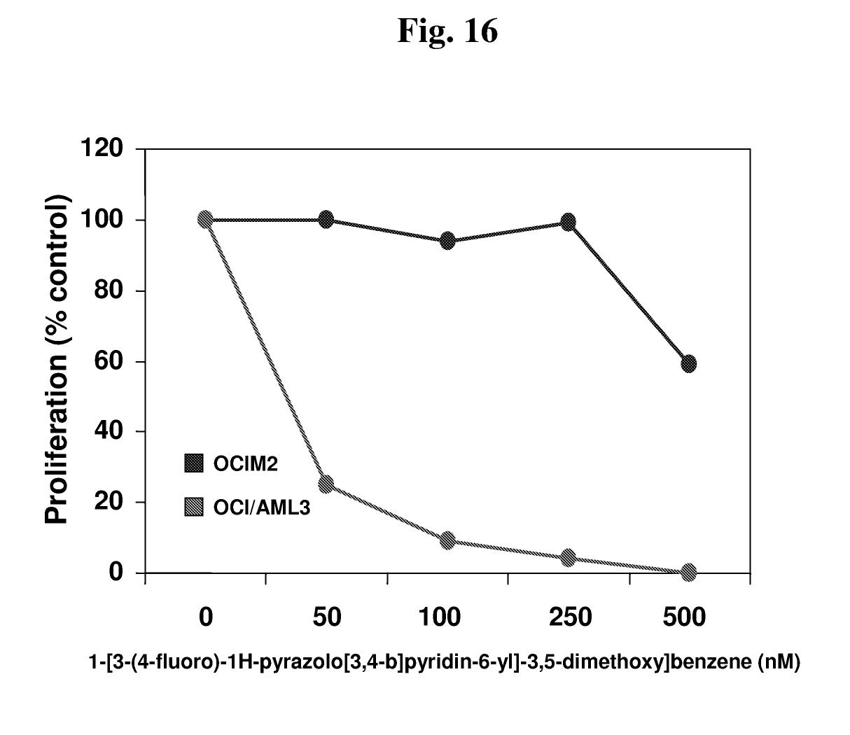

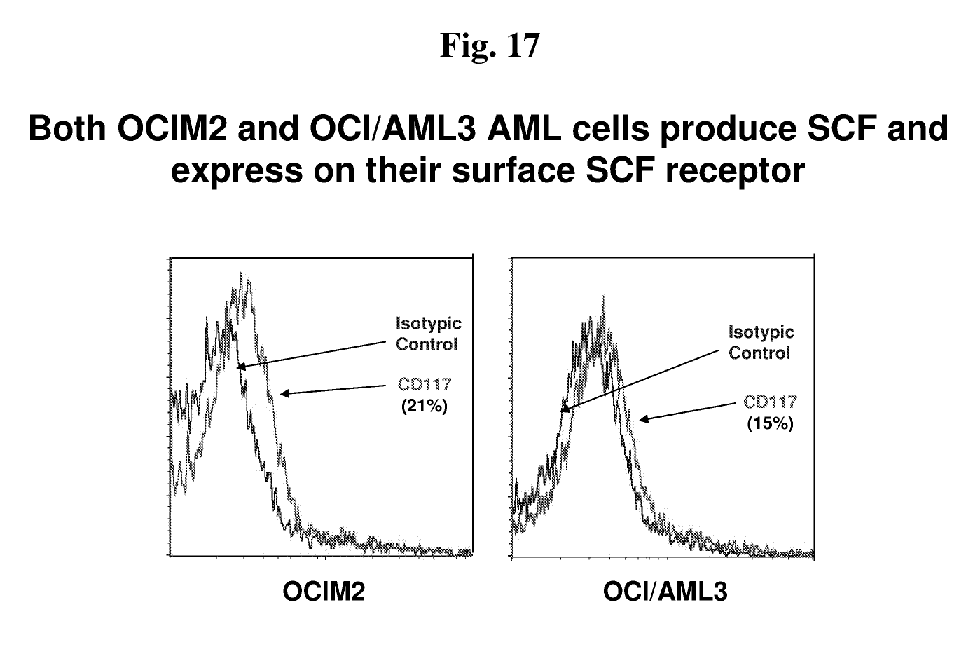

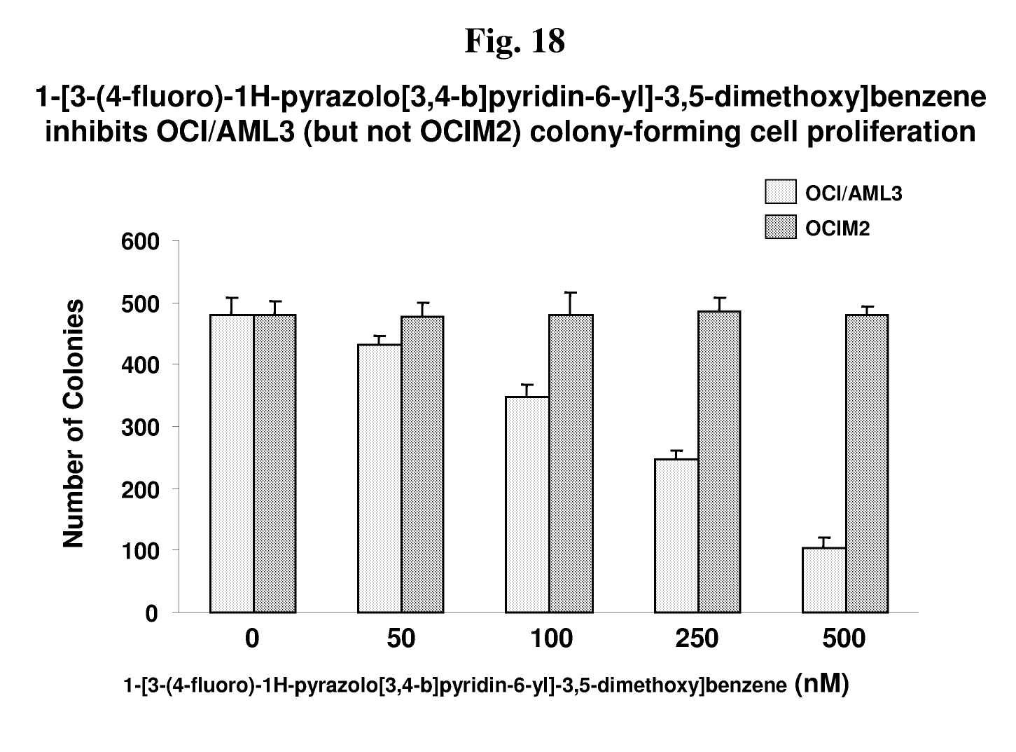

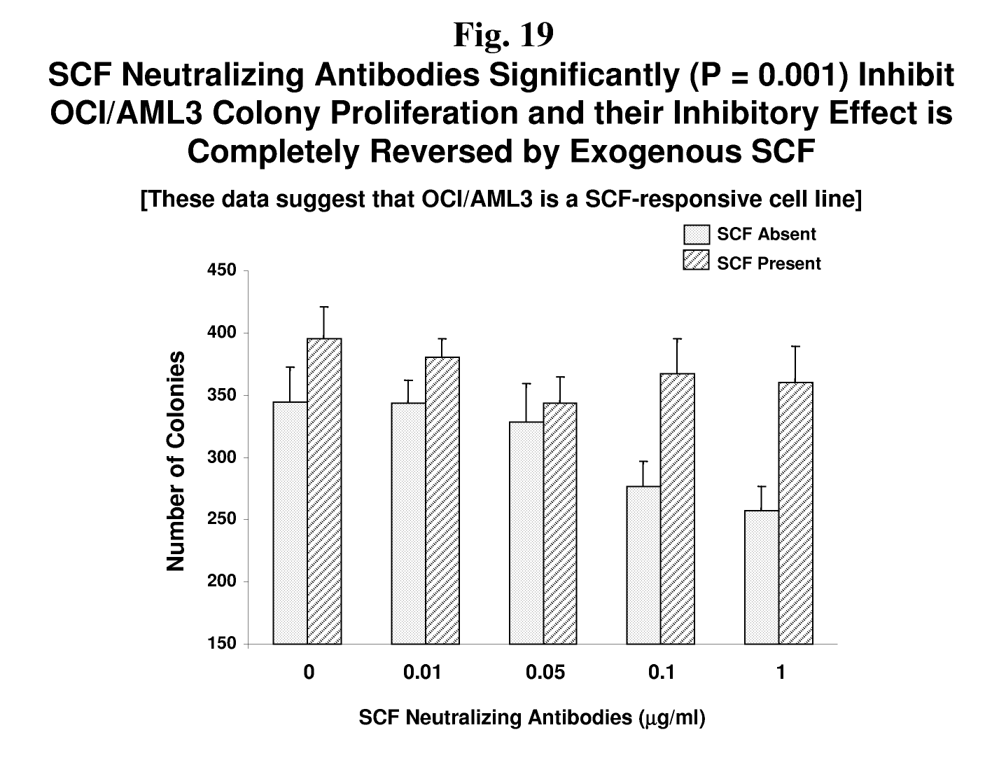

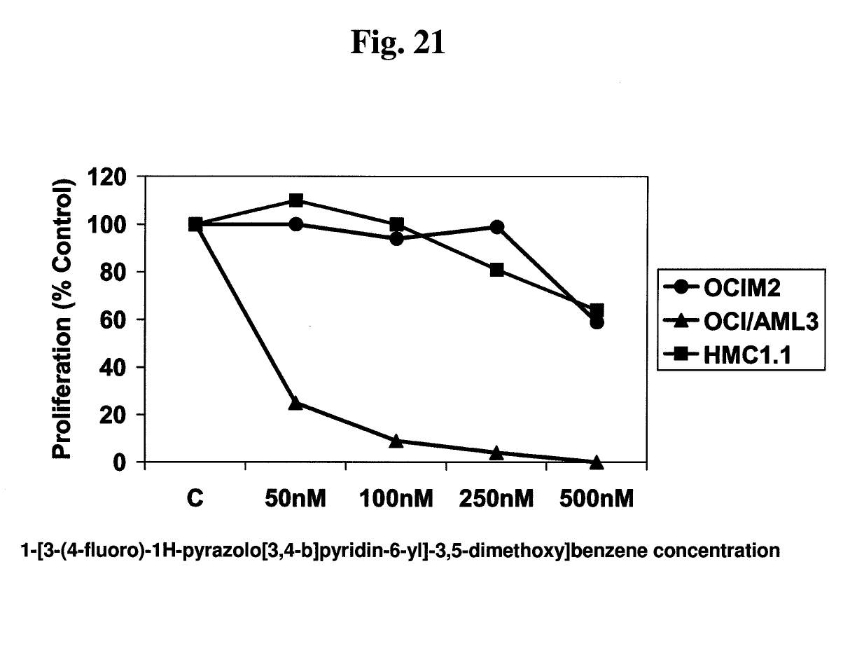

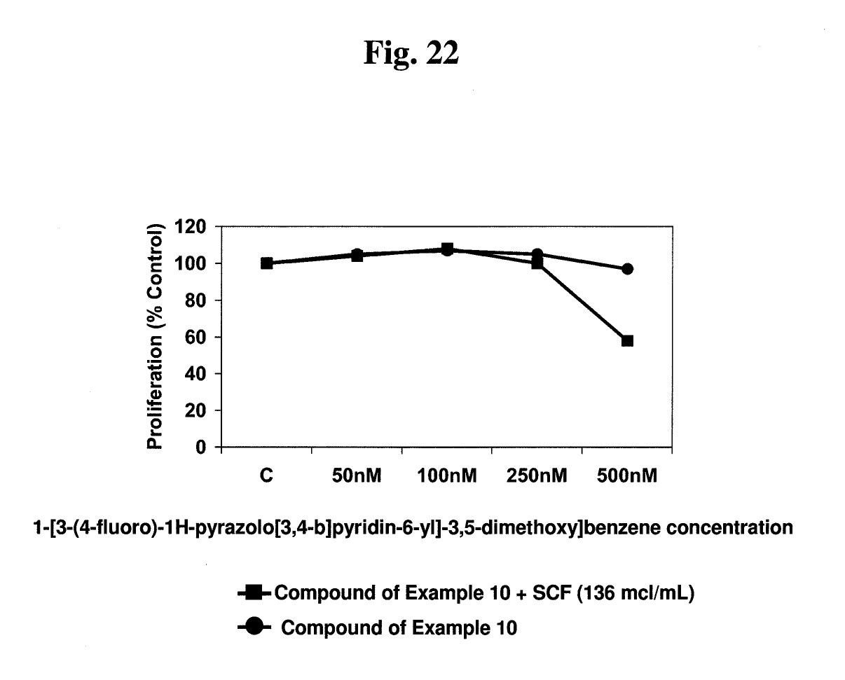

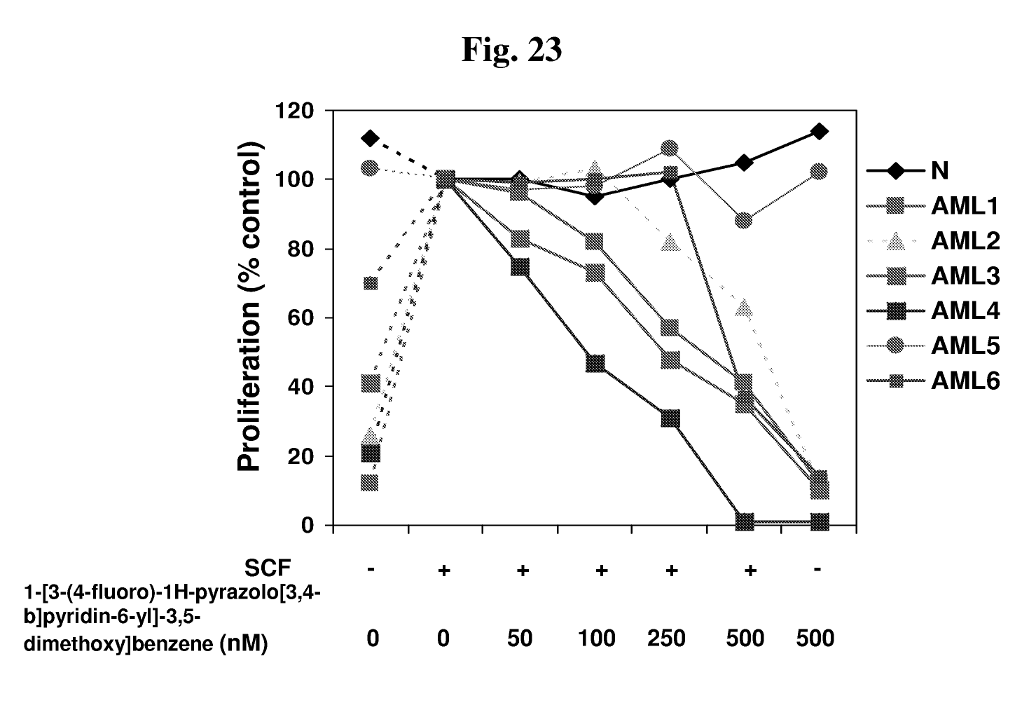

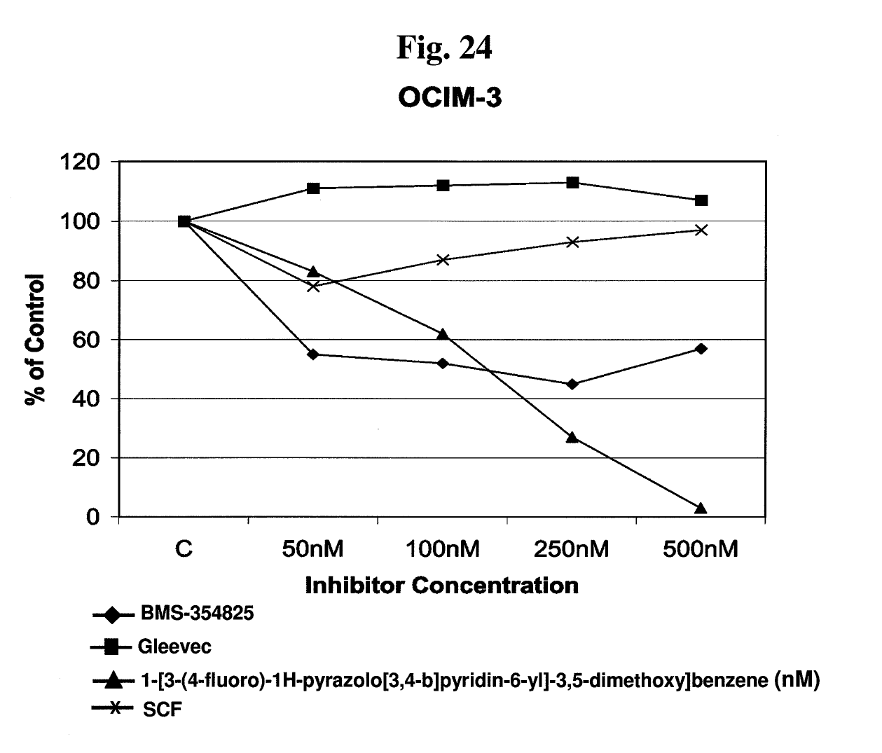

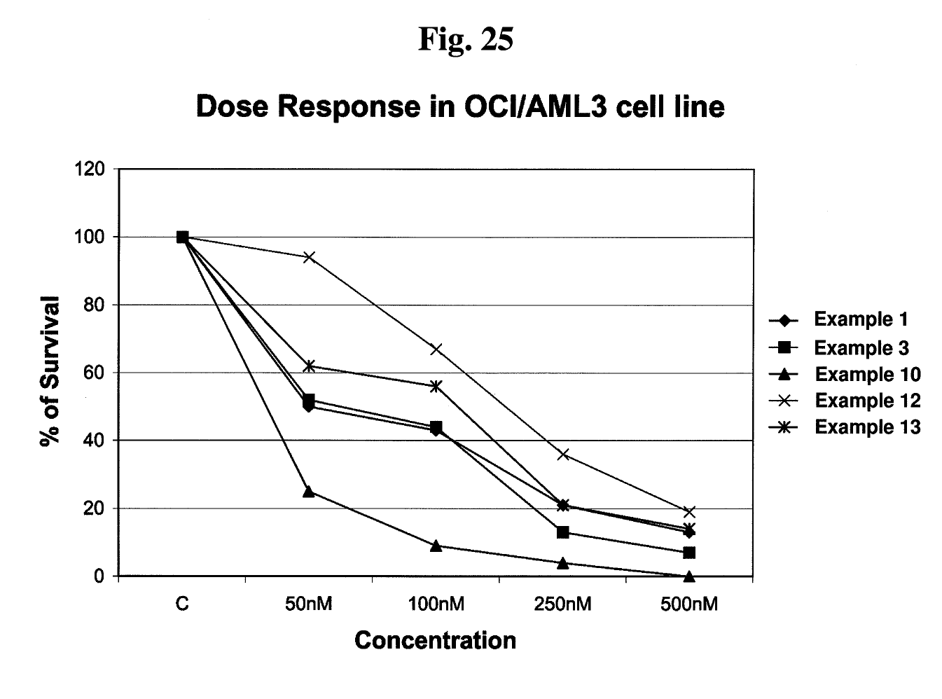

FIG. 16 shows the effect of the compound of Example 10 on proliferation in two different acute leukemia cell lines. Interestingly, OCI/AML3 is quite sensitive to the compound of Example 10 where OCIM2 is less sensitive. As shown in FIG. 17 both of these cell lines produce stem cell factor. This raised the question of why the two cell lines have differing sensitivities. FIG. 18 shows another experiment comparing the sensitivity of the two acute leukemia cell lines to the compound of Example 10 confirming the greater activity versus OCI/AML3. FIG. 19 shows that the presence of SCF neutralizing antibodies reverses the effects of the compound of Example 10 in OCI/AML3. By contrast OCI/M2 is not affected by the presence of stem cell factor neutralizing antibodies. This sheds some light on the differences between the two leukemia cell lines. FIG. 21 further shows the effect of the compound of Example 10 on additional cell line HMC1.1. Note that this cell line exhibits similar insenstivity as OCIM2. FIG. 22 shows that the effects of the compound of Example 10 are enhanced by the presence of externally introduced SCF. As shown in FIG. 23, in a series of cell lines AML5 is a unique cell line that exhibits less effective inhibition when treated with the compound of Example 10. As shown in FIG. 24, the compound of Example 10 shows favorable comparison in the OCIM cell line to known compounds BMS-354825 and Gleevec. It is also generally more effect than SCF alone. Related compounds in the family show the same effective ability to reduce OCI/AML3 cell line including, but not limited to the compounds of Examples 1, 3, 12 and 13, as shown in FIG. 25.

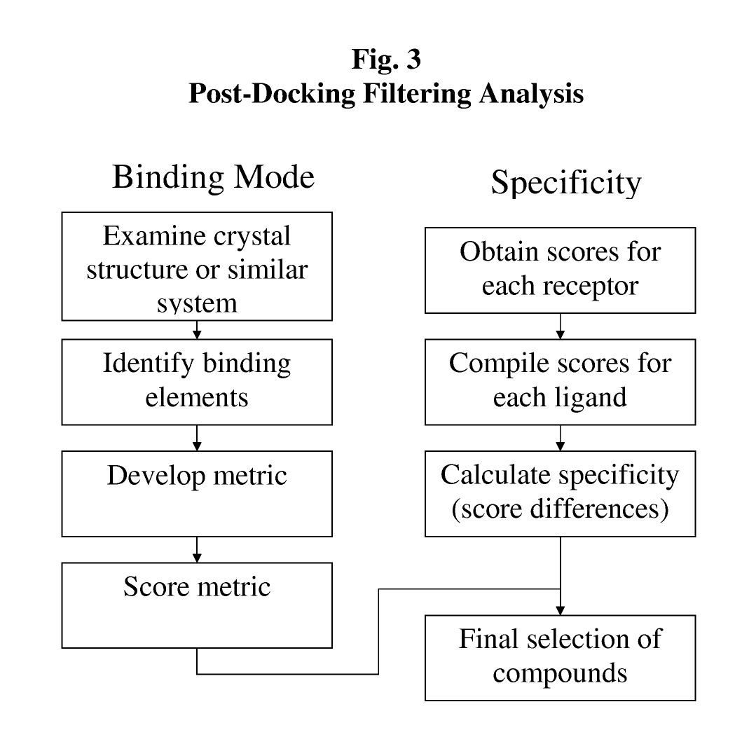

For the present study, the selectivity was compared in-silico against c-Met, a player in leukemia. Another element used in the selection of compounds was the evaluation of binding mode. Utilization of specific interactions along with the docking scores has been shown to increase the hit ratio of in-silico screening (Hindle, et al., (2002) J Comput Aided Mol Des 16, 129-49; Boehm, et al., (2002) Reviews in Computational Chemistry 18, 41-87). Information on these binding interactions was derived from an inhibitor bound crystal structure from Mol, et al. ((2004) Journal of Biological Chemistry 279:30, 31655-31663). An example of such a structure is provided by FIG. 2 which is a schematic showing GLEEVEC® bound to the ATP binding pocket of c-Kit. This level of analysis was not available as part of the normal docking interface, so code was written to analyze the data and provide consensus information. A flowchart showing this strategy is shown in FIG. 3. The analysis included identifying hydrogen bonding elements within each docking configuration for each ligand and comparing those against the specific areas of the receptor site. Those compounds within a particular distance were flagged and the combination of elements was used as a filter for the binding mode. This procedure was followed for docked configurations determined for each of the compounds in the screening libraries.

Several candidate compounds were identified in this manner. The high docking score of this compound may be due to an interaction such as hydrogen bonding with Thr670 of c-Kit. No group equivalent to Thr670, i.e., that provides the possibility of hydrogen bonding, appears to be present in MET. Therefore, compounds selected for study herein were designed to allow a possible interaction with Thr670. After restricting to a particular binding mode, the above mentioned filters were then applied in the order of docking score, consensus score and, lastly, for selectivity. This computational strategy indicated that compounds based on a 1H-pyrazolo[3,4-b]pyridine core may be effective for inhibition of c-Kit kinase.

We claim the following:

1. A compound of the structural Formula I 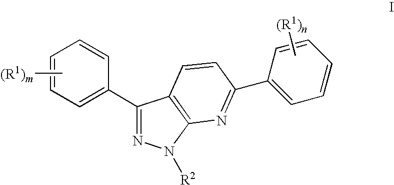
wherein m and n is an integer from 1 to 5;
R¹ is independently halogen, alkyl, alkoxycarbonyl, alkoxy, hydrogen, or cyano, any of which may be optionally substituted;
R² is selected from the group consisting of hydrogen, acyl, alkenyl, alkoxyalkyl, alkoxycarbonyl, alkoxycarbonylalkyl, alkyl, alkylaminocarbonyl, alkylcarbonylalkyl, alkylthioalkyl, alkylsulfinylalkyl, alkynyl, aminoalkyl, aminocarbonylalkyl, aryl, arylsulfonyl, aralkyl, carboxyalkyl, cycloalkyl, haloalkyl, heteroaryl, heteroaralkyl, or hydroxyalkyl, any of which may be optionally substituted; and
R³ is independently hydrogen, acetamido, acyl, alkenyl, alkoxyalkyl, alkoxycarbonyl, alkoxycarbonylalkyl, alkyl, alkylaminocarbonyl, alkylcarbonylalkyl, alkylthioalkyl, alkylsulfinylalkyl, alkynyl, aminoalkyl, aminocarbonyl, aminocarbonylalkyl, aryl, arylsulfonyl, arylcarbonyl, fused pyrrole, aralkyl, carboxyalkyl, cycloalkyl, haloalkyl, heteroaryl, heteroaralkyl, hydroxyalkyl, or phenol any of which may be optionally substituted;
wherein when n=2, R³ is not CONHEt and Me; and
when n=3, R³ is not F, Me, and CONHEt;
F, Me and 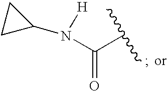
F, Me and

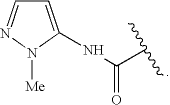

2. A compound of the structural Formula II: 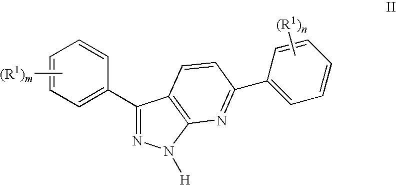
wherein m and n is an integer from 1 to 5;
R¹ is independently hydrogen, halogen, alkyl, ester, alkoxy, hydrogen, or cyano, any of which may be optionally substituted; and
R² is independently hydrogen, acetamido, acyl, alkenyl, alkoxyalkyl, alkoxycarbonyl, alkoxycarbonylalkyl, alkyl, alkylaminocarbonyl, alkylcarbonylalkyl, alkylthioalkyl, alkylsulfinylalkyl, alkynyl, aminoalkyl, aminocarbonyl, aminocarbonylalkyl, aryl, arylsulfonyl, arylcarbonyl, fused pyrrole, aralkyl, carboxyalkyl, cycloalkyl, haloalkyl, heteroaryl, heteroaralkyl, hydroxyalkyl, or phenol any of which may be optionally substituted;
wherein when n=2, R² is not CONHEt and Me; and
when n=3, R² is not F, Me, and CONHEt;
F, Me and 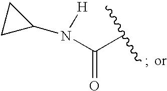
F, Me and

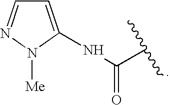

3. The compound as recited in claim 2 wherein
R¹ is independently selected from the group consisting of aryl or heteroaryl, optionally substituted by 1-3 substituents independently selected from the group consisting of acyl, acylamino, alkoxy, alkoxycarbonyl, alkyl, alkylaminocarbonyl, cyano, halo, haloalkyl, heteroaryl, heterocyclo, heterocyclocarbonyl, hydroxy; and
R² is independently selected from the group consisting of aryl or heteroaryl, acyl, acylamino, alkoxy, alkoxycarbonyl, alkyl, alkylaminocarbonyl, cyano, halo, haloalkyl, heteroaryl, heterocyclo, heterocyclocarbonyl, hydroxyl,
provided that R² is not para-substituted CH₃ and acylamino, para-substituted CH₃ and alkylaminocarbonyl, para-substituted C₁ and acylamino, para-substituted C₁ and alkylaminocarbonyl.

4. A compound selected from the group consisting of Example 1 to Example 56.

5. A compound or composition as recited in claim 1 for use as a medicament.

6. A compound or composition as recited in claim 1 for use in the manufacture of a medicament for the prevention or treatment of a disease or condition ameliorated by the inhibition of c-Kit.

7. A pharmaceutical composition as recited in claim 1 and claim 2 useful for the treatment or prevention of a c-Kit-mediated disease.

8. A pharmaceutical composition comprising a compound as recited in claim 1 or claim 2 together with a pharmaceutically acceptable carrier.

9. A method of inhibition of c-Kit comprising contacting c-Kit with a compound as recited in claim 1 or claim 2.

10. A method of treatment of a c-Kit-mediated disease comprising the administration of a therapeutically effective amount of the compound as recited in claim 1 or claim 2 to a patient in need thereof.

11. The method as recited in claim 10 above wherein the disease is cancer.

12. A method of treatment of c-Kit-mediated disease comprising the administration of:
a. a therapeutically effective amount of a compound as recited in claim 1 or claim 2, and
b. another therapeutic agent.

13. A method for achieving an effect in a patient comprising the administration of a therapeutically effective amount of a compound as recited in claim 1 or claim 2 to a patient, wherein the effect is selected from the group consisting Example 1 through Example 56.
